SEQUENCE LISTING (10 sequences)

SEQ ID NO: 1 - protein, 28 aa, synthetic construct
  REGION 1..28: note = NAT_29
KKGSRSCFFIYLDRGTKKGSRSCFFYLP

SEQ ID NO: 2 - protein, 121 aa, synthetic construct
  REGION 1..121: note = ID3166
AFLFTSTGVPKKAAEAAFFLYLNKGTKKGSRSCFFIYLDRGTKKGSRSCFFYLPRQGYQKGSRGCFFIYLDRGTKKGSRGCFFIYLDCEKRAGNVCIRKCRGRYLHKKTPRRYRNAEATCS

SEQ ID NO: 3 - protein, 7 aa, synthetic construct
  REGION 1..7: note = NAT_29 motif
  REGION 3..4: note = misc_feature - Xaa can be any naturally occurring amino acid
SCXXYLP

SEQ ID NO: 4 - protein, 8 aa, synthetic construct
  REGION 1..8: note = NAT_29 motif
  REGION 3..5: note = misc_feature - Xaa can be any naturally occurring amino acid
SCXXXYLD

SEQ ID NO: 5 - protein, 7 aa, synthetic construct
  REGION 1..7: note = NAT_29 motif
SCFFYLP

SEQ ID NO: 6 - protein, 12 aa, synthetic construct
  REGION 1..12: note = NAT_29 motif
KKGSRSCFFYLP

SEQ ID NO: 7 - protein, 8 aa, synthetic construct
  REGION 1..8: note = NAT_29 motif
SCFFIYLD

SEQ ID NO: 8 - protein, 12 aa, synthetic construct
  REGION 1..12: note = KGSRSCFFIYLD
KGSRSCFFIYLD

SEQ ID NO: 9 - protein, 28 aa, synthetic construct
  REGION 1..28: note = NAT_38
KKGSRSCFFYLPRQGYQKGSRGCFFIYL

SEQ ID NO: 10 - DNA, 84 bp, synthetic construct
  misc_feature 1..84: note = NAT_29
  source 1..84: mol_type = other DNA
aaaaaaggcagcagaagctgcttttttatttacctcgacaggggtaccaaaaaaggcagcagaagctgctttttttatttacct

(19) United States
(12) Patent Application Publication
(10) Pub. No.: US 2024/0327471 A1
(43) Pub. Date: Oct. 3, 2024

(54) IL-10 INDUCING POLYPEPTIDES AND USES THEREOF

(71) Applicant: ENTEROME S.A., PARIS (FR)

(72) Inventors: Christophe BONNY, PARIS (FR); Antonietta CULTRONE, L'Hay Les Roses (FR); Helene PFISTER, PARIS (FR)

(21) Appl. No.: 18/291,050

(22) PCT Filed: Jul. 26, 2022
(86) PCT No.: PCT/EP2022/070987

§ 371 Date: Jan. 22, 2024

(30) Foreign Application Priority Data
Jul. 27, 2021 (EP) 21306045.2

(51) Int. Cl. C07K 14/245; A61K 38/00; A61P 1/00; A61P 29/00; C12N 1/20; C12N 15/70

(52) U.S. Cl. CPC C07K 14/245; A61P 1/00; A61P 29/00; C12N 1/20; C12N 15/70; A61K 38/00

(57) ABSTRACT

The present invention relates to polypeptides, which are capable of inducing and/or enhancing secretion of IL-10 from human cells. Accordingly, the present invention provides polypeptides, which stimulate IL-10 release from human immune cells. The present invention also relates to nucleic acids encoding such polypeptide, cells expressing such polypeptide, respective pharmaceutical compositions and uses thereof.

The present invention relates to polypeptides, which are capable of inducing and/or enhancing secretion of IL-10 from human cells. The present invention also relates to nucleic acids encoding such polypeptides, cells expressing such polypeptides, respective pharmaceutical compositions and uses thereof.

The human intestinal microbiota (HIM) is a huge and complex community of 1014 bacteria which colonize the human gastrointestinal tract (GIT) and which is now considered as a hidden human organ. HIM has essential functions such as maintaining intestinal homeostasis, inhibiting the growth of pathogens, producing antimicrobial compounds and improving the intestinal barrier function. A number of evidences support the fact that several GI disorders including inflammatory bowel diseases (IBD), non-alcoholic steatohepatitis (NASH), alcoholic liver disease and even brain activity (gut brain axis) could be the result of an altered balance (dysbiosis) of the intestinal microbiota causing an abnormal production of particular metabolites and proteins. Over the last decade a huge amount of literature has described the identification of bacterial metabolites and their potential effects on human health (Levy M, Thaiss CA, Elinav E. Metabolites: messengers between the microbiota and the immune system. Genes Dev. 2016;30 (14): 1589-97; Guo C J, Chang F Y, Wyche T P, Backus K M, Acker T M, Funabashi M, Taketani M, Donia M S, Nayfach S, Pollard K S, Craik C S, Cravatt B F, Clardy J, Voigt C A, Fischbach M A. Discovery of Reactive Microbiota-Derived Metabolites that Inhibit Host Proteases. Cell. 2017;168 (3): 517-526.e18).

Currently, studies of interactions between bacterial proteins with human gut cells are emerging (Weigele B A, Orchard R C, Jimenez A, Cox G W, Alto N M. A systematic exploration of the interactions between bacterial effector proteins and host cell membranes. Nat Commun. 2017;8 (1): 532; Guven-Maiorov E, Tsai C J, Nussinov R. Structural host-microbiota interaction networks. PLOS Comput Biol. 2017;13 (10): e1005579). Microbial proteins may have the ability to interact with many human receptors, such as GPCRs, kinase receptors and transporters and may affect many different signalling pathways involved in immune surveillance, metabolism and cellular integrity. Molecular mimicry between human and microbial proteins have been proposed: E. coli protein ClpB, mimics the human alpha MSH, a neuropeptide playing a key role in signaling satiation (Breton J, Tennoune N, Lucas N, Francois M, Legrand R, Jacquemot J, Goichon A, Guerin C, Peltier J, Pestel-Caron M, Chan P, Vaudry D, do Rego J C, Liénard F, Pénicaud L, Fioramonti X, Ebenezer IS, Hökfelt T, Déchelotte P, Fetissov S O (2016) Gut Commensal E. coli Proteins Activate Host Satiety Pathways following Nutrient-Induced Bacterial Growth. Cell Metab. 2016 Feb. 9;23 (2): 324-34); the Helicobacter Pylori CagA interacts with human tumor suppressor TP53BP2 (Buti L, Spooner E, Van der Veen A G, Rappuoli R, Covacci A, Ploegh H L. (2011) Helicobacter pylori cytotoxin-associated gene A (CagA) subverts the apoptosis-stimulating protein of p53 (ASPP2) tumor suppressor pathway of the host. Proc Natl Acad Sci U S A. 2011 May 31;108 (22): 9238-43); SLPA from Lactobacillus acidophilus, is a DC-SIGN ligand functionally involved in the modulation of DCs and T cells functions (Konstantinov S R, Smidt H, de Vos W M, Bruijns S C, Singh S K, Valence F, Molle D, Lortal S, Altermann E, Klaenhammer T R, van Kooyk Y. (2008) S layer protein A of Lactobacillus acidophilus NCFM regulates immature dendritic cell and T cell functions. Proc Natl Acad Sci U S A. 2008 Dec. 9;105 (49): 19474-9); FAp2 from Fusobacterium nucleatum has been shown to mediate Colorectal Adenocarcinoma enrichment by binding to tumor-expressed Gal-GalNAc, and to bind to the TIGIT receptor (Gur C, Ibrahim Y, Isaacson B, Yamin R, Abed J, Gamliel M, Enk J, Bar-On Y, Stanietsky-Kaynan N, Coppenhagen-Glazer S, Shussman N, Almogy G, Cuapio A5, Hofer E, Mevorach D, Tabib A, Ortenberg R, Markel G, Miklić K, Jonjic S, Brennan C A, Garrett W S, Bachrach G, Mandelboim O. (2015) Binding of the Fap2 protein of Fusobacterium nucleatum to human inhibitory receptor TIGIT protects tumors from immune cell attack. Immunity. 2015 Feb. 17;42 (2): 344-355).

Thus, it is possible that proteins from commensal bacteria may have therapeutic applications by mimicking host proteins and interacting directly with the host cells and the immune system.

Interleukin-10 (IL-10) is a key immunoregulatory cytokine produced by various cell types, such as lymphocytes, monocytes, macrophages, mast cells and intestinal epithelial cells (Shouval D S, Biswas A, Goettel J A, McCann K, Conaway E, Redhu N S, Mascanfroni I D, Al Adham Z, Lavoie S, Ibourk M, Nguyen D D, Samsom J N, Escher JC, Somech R, Weiss B, Beier R, Conklin L S, Ebens CL, Santos F G, Ferreira A R, Sherlock M, Bhan A K, Müller W, Mora J R, Quintana F J, Klein C, Muise A M, Horwitz B H, Snapper S B (2014) Interleukin-10 receptor signaling in innate immune cells regulates mucosal immune tolerance and anti-inflammatory macrophage function. Immunity. 2014 May 15;40 (5): 706-19) with the major functions of limiting mucosal immune responses and maintaining gut homeostasis.

IL-10 is often considered as the “prototype of anti-inflammatory cytokines” and it is known to downregulate the expression of Th1 cytokines, MHC class II antigens, and co-stimulatory molecules on macrophages. Its inhibitory action is exerted mainly against the most typical markers of inflammation such as IL-1, IL-6, TNF-α, GM-CSF and TNF-γ. The three major pro-inflammatory cytokines IL-1β, IL-6, and TNF-α are responsible for the onset and the maintenance of the inflammatory state in many diseases. While IL-10 is able to inhibit both the Th1-type and the Th2-type responses, the effect on Th1 subpopulation is predominant and IL-10 is considered as a promoter of the Th2 response (pleiotropic effect). In addition, IL-10 also enhances B cell survival, proliferation, antibody production as well as the production of anti-inflammatory factors including soluble TNF-α receptors and IL-1RA.

Polymorphisms in the IL-10 locus confer the risk for ulcerative colitis and Crohn's disease (Franke A, Balschun T, Karlsen T H, Sventoraityte J, Nikolaus S, Mayr G, Domingues F S, Albrecht M, Nothnagel M, Ellinghaus D, Sina C, Onnie C M, Weersma R K, Stokkers P C, Wijmenga C, Gazouli M, Strachan D, McArdle WL, Vermeire S, Rutgeerts P, Rosenstiel P, Krawczak M, Vatn M H; IBSEN study group, Mathew CG, Schreiber S (2008) Sequence variants in IL10, ARPC2 and multiple other loci contribute to ulcerative colitis susceptibility. Nat Genet. 2008 November;40 (11): 1319-23; Franke A, McGovern D P, Barrett J C, . . . . Parkes M. (2010) Genome-wide meta-analysis increases to 71 the number of confirmed Crohn's disease susceptibility loci. Nat Genet. 2010 December;42 (12): 1118-25), and mice and humans deficient in either IL-10 or IL-10 receptor (IL-10R) exhibit severe intestinal inflammation, a process dominated by a T helper type 1 and type 17 immune response and marked proinflammatory cytokines secretion (Begue B, Verdier J, Rieux-Laucat F, Goulet O, Morali A, Canioni D, Hugot J P, Daussy C, Verkarre V, Pigneur B, Fischer A, Klein C, Cerf-Bensussan N, Ruemmele F M. (2011) Defective IL 10 signaling defining a subgroup of patients with inflammatory bowel disease. Am J Gastroenterol. Aug;106 (8): 1544-55; Moran C J, Walters T D, Guo C H, Kugathasan S, Klein C, Turner D, Wolters V M, Bandsma R H, Mouzaki M, Zachos M, Langer J C, Cutz E, Benseler S M, Roifman C M, Silverberg M S, Griffiths A M, Snapper S B, Muise A M. (2013) IL-10R polymorphisms are associated with very-early-onset ulcerative colitis. Inflamm Bowel Dis. Jan; 19 (1): 115-23).

In view thereof, recombinant human IL-10 has emerged as candidate drug in acute and chronic inflammatory diseases, autoimmune disorders and allergic diseases, rejection of transplanted organs and graft-versus-host diseases after transplantation, and infectious diseases. However, studies with high-dose recombinant human IL-10 revealed several problems relating to the side effects of high-dose recombinant human IL-10 and the development of antibodies against recombinant human IL-10, restricting its usefulness in therapeutic applications (Fioranelli M. 2014, Twenty-five years of studies and trials for the therapeutic application of IL-10 immunomodulating properties. From high doses administration to low dose medicine new paradigm. J Integr Cardiol 1: DOI: 10.15761/JIC.1000102).

Accordingly, there is a need to provide increased IL-10 levels in therapeutic applications by means other than administration of high-dose recombinant human IL-10. In view thereof, it is the object of the present invention to provide compounds, which are capable of inducing secretion of (endogenous) IL-10.

This object is achieved by means of the subject-matter set out below, in particular in the items provided by the present invention and in the appended claims.

### ITEMS OF THE INVENTION

The present invention provides in particular the following items:
1. A polypeptide comprising or consisting of an amino acid sequence according to general formula (I):

(I)
SCX₁X₂X₃YLX₄

wherein
X₁ may be any amino acid,
X₂ may be any amino acid,
X₃ may be any amino acid or is deleted, and
X₄ is P or D.
2. The polypeptide according to item 1 comprising or consisting of an amino acid sequence according to general formula (Ia):

(Ia)
KGSRSCX₁X₂X₃YLX₄

wherein X₁-X₄ are defined as in item 1.
3. The polypeptide according to item 1 or 2, wherein X₃ is deleted and X₄ is P.
4. The polypeptide according to item 3 comprising or consisting of the amino acid sequence according to SEQ ID NO: 5 or 6. [SCFFYLP or KKGSRSCFFYLP]
5. The polypeptide according to item 1 or 2, wherein X₃ may be any amino acid and X₄ is D.
6. The polypeptide according to item 5 comprising or consisting of the amino acid sequence according to SEQ ID NO: 7 or 8. [SCFFIYLD or KGSRSCFFIYLD]
7. The polypeptide according to any one of the previous items comprising two cysteine residues, optionally forming a disulfide bond.
8. The polypeptide according to any one of the previous items comprising two amino acid sequences according to any one of items 1 to 6, wherein the two amino acid sequences may be the same or different.
9. A polypeptide comprising or consisting of an amino acid sequence according to general formula (II):

(II)
CX₁X₂X₃YLX₄X₅X₆X₇X₈X₉KGSRX₁₀C

wherein
X₁ may be any amino acid,
X₂ may be any amino acid,
X₃ may be any amino acid or is deleted,
X₄ is Por D,
X₅ may be any amino acid,
X₆ may be any amino acid or is deleted,
X₇ may be any amino acid,
X₈ may be any amino acid,
X₉ is K or Q, and
X₁₀ may be any amino acid.
10. The polypeptide according to any one of the previous items comprising or consisting of an amino acid sequence according to general formula (IIa):

(IIa)
SCFFX₃YLX₄RX₆GX₈X₉KGSRX₁₀C

wherein
X₃ is I or deleted,
X₄ is Por D,
X₆ is Q or deleted,
X₈ is Tor Y,
X₉ is K or Q, and
X₁₀ is S or G.
11. The polypeptide according to item 9 or 10, wherein either X₃ or X₆, but not X₃ and X₆, are deleted.
12. The polypeptide according to any one of the previous items having a length of at least 17 amino acids.
13. A polypeptide comprising or consisting of an amino acid sequence according to SEQ ID NO: 1, wherein, optionally, 0, 1, 2, 3, 4, 5, 6, 7 or 8 amino acids may be substituted, added and/or deleted, with the proviso that the serine residue at position 6 of SEQ ID NO: 1 is maintained.
14. The polypeptide according to item 13 comprising an amino acid sequence as defined in any one of the previous items.
15. The polypeptide according to item 13 or 14, wherein the cysteine residue(s) at position 7 and/or 23 of SEQ ID NO: 1 is/are maintained.
16. The polypeptide according to any one of the previous items, wherein the polypeptide does not comprise an amino acid sequence according to SEQ ID NO: 2.
17. The polypeptide according to any one of the previous items, wherein the polypeptide has a length of no more than 120 amino acids.
18. The polypeptide according to any one of the previous items, wherein the polypeptide is capable of inducing and/or enhancing IL-10 secretion from human cells.
19. The polypeptide according to item 18, wherein the human cells are human immune cells, preferably PBMCs.
20. The polypeptide according to item 18 or 19, wherein the human cells are monocytes.
21. The polypeptide according to any one of items 18 to 20, wherein IL-10 secretion from human cells stimulated with said polypeptide is the same or higher than IL-10 secretion stimulated with lipopolysaccharide (LPS), e.g. with 10 ng/ml or 100 ng/ml LPS.
22. The polypeptide according to any one of the previous items, wherein the polypeptide comprises or consists of an amino acid sequence according to SEQ ID NO: 1.
23. The polypeptide according to any one of the previous items, wherein the polypeptide comprises or consists of an amino acid sequence according to SEQ ID NO: 9.
24. A nucleic acid comprising a polynucleotide encoding the polypeptide according to any one of the previous items.
25. The nucleic acid according to item 24, wherein the nucleic acid is a DNA molecule or an RNA molecule; preferably selected from genomic DNA; cDNA; siRNA; rRNA; mRNA; antisense DNA; antisense RNA; ribozyme; complementary RNA and/or DNA sequences; RNA and/or DNA sequences with or without expression elements, regulatory elements, and/or promoters; a vector; and combinations thereof.
26. The nucleic acid according to item 24 or 25, wherein the nucleic acid sequence of the polynucleotide encoding the polypeptide, shares at least 80% sequence identity with SEQ ID NO: 10.
27. The nucleic acid according to any one of items 24 to 26, wherein the polynucleotide encoding the polypeptide is codon-optimized for expression by prokaryotic cells, preferably bacteria.
28. An expression cassette comprising a polynucleotide encoding the polypeptide according to any one of items 1 to 23 and, operably linked thereto, a regulatory element, preferably for expression in a prokaryotic cell, such as a bacterium.
29. The expression cassette according to item 28 comprising a regulatory element for heterologous expression and/or overexpression of the encoded polypeptide.
30. A vector comprising the nucleic acid according to any one of items 24 to 27 or the expression cassette according to item 28 or 29.
31. A (host) cell expressing the polypeptide according to any one of items 1 to 23; or comprising the nucleic acid according to any one of items 24 to 27, the expression cassette according to item 28 or 29, or the vector according to item 30.
32. The (host) cell according to item 31, wherein the (host) cell is a bacterium, preferably a genetically engineered bacterium.
33. A genetically engineered bacterium capable of inducing and/or enhancing IL-10 secretion from a human cell, the bacterium comprising the nucleic acid according to any one of items 24 to 27, the expression cassette according to item 28 or 29, or the vector according to item 30.
34. A genetically engineered bacterium (over) expressing the polypeptide according to any one of items 1 to 23.
35. A culture medium comprising the polypeptide according to any one of items 1 to 23, the (host) cell according to item 31 or 32, or the bacterium according to item 33 or 34.
36. The culture medium according to item 35 further comprising a (self) antigen.
37. An isolated human cell cultured with the culture medium according to item 35 or 36.
38. The cell according to item 37, wherein the cell is a human immune cell, preferably a PBMC.

39. The cell according to item 37 or 38, wherein the cell is a monocyte, a macrophage, a dendritic cell or a lymphocyte, preferably a T lymphocyte.
40. A pharmaceutical composition comprising the polypeptide according to any one of items 1 to 23, the nucleic acid according to any one of items 24 to 27, the vector according to item 30, the cell according to item 31 or 32, the bacterium according to item 33 or 34, or the human cell according to any one of items 37 to 39 and, optionally, a pharmaceutically acceptable excipient or carrier.
41. The polypeptide according to any one of items 1 to 23, the nucleic acid according to any one of items 24 to 27, the vector according to item 30, the cell according to item 31 or 32, the bacterium according to item 33 or 34, the human cell according to any one of items 37 to 39, or the pharmaceutical composition according to item 40 for use in medicine.
42. The polypeptide, the nucleic acid, the vector, the cell, the bacterium, the human cell or the pharmaceutical composition for use according to item 41 in treating an inflammatory disease or an autoimmune disorder.
43. The polypeptide, the nucleic acid, the vector, the cell, the bacterium, the human cell or the pharmaceutical composition for use according to item 41 in treating inflammatory bowel disease (IBD).
44. The polypeptide, the nucleic acid, the vector, the cell, the bacterium, the human cell or the pharmaceutical composition for use according to item 41 in treating an allergy.
45. A method for reducing, treating, alleviating symptoms of or ameliorating an inflammatory disease or an autoimmune disorder in a subject, comprising the step of administering to said subject the polypeptide according to any one of items 1 to 23, the nucleic acid according to any one of items 24 to 27, the vector according to item 30, the cell according to item 31 or 32, the bacterium according to item 33 or 34, the human cell according to any one of items 37 to 39, or the pharmaceutical composition according to item 40.
46. A method for inducing and/or enhancing IL-10 secretion in a subject, comprising the step of administering to said subject the polypeptide according to any one of items 1 to 23, the nucleic acid according to any one of items 24 to 27, the vector according to item 30, the cell according to item 31 or 32, the bacterium according to item 33 or 34, the human cell according to any one of items 37 to 39, or the pharmaceutical composition according to item 40.
47. A method for inducing tolerance in a subject, comprising the step of administering to said subject the polypeptide according to any one of items 1 to 23, the nucleic acid according to any one of items 24 to 27, the vector according to item 30, the cell according to item 31 or 32, the bacterium according to item 33 or 34, the human cell according to any one of items 37 to 39, or the pharmaceutical composition according to item 40.

The invention, and in particular the items outlined above, are described in more detail below.

### DEFINITIONS

Unless otherwise defined herein, scientific and technical terms used in the present application shall have the meanings that are commonly understood by those of ordinary skill in the art. Further, unless otherwise required by context, nomenclatures used herein, and techniques of cell and tissue culture are those well-known and commonly used in the art.

Such techniques are fully explained in the literature, such as Owen et al. (Kuby Immunology, 7^{th}, edition, 2013-W. H. Freeman) and Sambrook et al. (Molecular cloning: A laboratory manual 4th edition, Cold Spring Harbor Laboratory Press-Cold Spring Harbor, NY, USA, 2012).

Nevertheless, with respect to the use of different terms throughout the current specification, the following definitions more particularly apply.

The term “(poly) peptide” as used herein refers to a peptide and/or to a polypeptide. The terms “peptide”, “polypeptide”, “protein” and variations of these terms refer to peptides, oligopeptides, polypeptides, or proteins comprising at least two amino acids joined to each other preferably by a normal peptide bond, or, alternatively, by a modified peptide bond, such as for example in the cases of isosteric peptides. In particular, the terms “peptide”, “polypeptide” and “protein” refer to a sequential chain of amino acids of any length linked together via peptide bonds (—NHCO—). Peptides, polypeptides and proteins can play a structural and/or functional role in a cell in vitro and/or in vivo. The terms “peptide”, “polypeptide”, “protein” preferably encompass amino acids chains in size ranging from 2 to at least about 1000 amino acid residues. The term “peptide” preferably encompasses herein amino acid chains in size of less than about 30 amino acids, while the terms “polypeptide” and “protein” preferably encompass amino acid chains in size of at least 30 amino acids. The terms “polypeptide” and “protein” are used herein interchangeably. In some embodiments, the terms “peptide”, “polypeptide”, “protein” also include “peptidomimetics” which are defined as peptide analogs containing non-peptidic structural elements, which peptides are capable of mimicking or antagonizing the biological action(s) of a natural parent peptide. A peptidomimetic lacks classical peptide characteristics such as enzymatically scissile peptide bonds. In particular, a peptide, polypeptide or protein can comprise amino acids other than the 20 amino acids defined by the genetic code in addition to these amino acids, or it can be composed of amino acids other than the 20 amino acids defined by the genetic code. In particular, a peptide, polypeptide or protein in the context of the present invention can equally be composed of amino acids modified by natural processes, such as post-translational maturation processes or by chemical processes, which are well known to a person skilled in the art. Such modifications are fully detailed in the literature. These modifications can appear anywhere in the polypeptide: in the peptide skeleton, in the amino acid chain or even at the carboxy-or amino-terminal ends. In particular, a peptide or polypeptide can be branched following an ubiquitination or be cyclic with or without branching. This type of modification can be the result of natural or synthetic post-translational processes that are well known to a person skilled in the art. The terms “peptide”, “polypeptide”, “protein” in the context of the present invention in particular also include modified peptides, polypeptides and proteins. For example, peptide, polypeptide or protein modifications can include acetylation, acylation, ADP-ribosylation, amidation, covalent fixation of a nucleotide or of a nucleotide derivative, covalent fixation of a lipid or of a lipidic derivative, the covalent fixation of a phosphatidylinositol, covalent or non-covalent cross-linking, cyclization, disulfide bond formation, demethylation, glycosylation including pegylation, hydroxylation, iodization, methylation, myristoylation, oxidation, proteolytic processes, phosphorylation, prenylation, racemization, seneloylation, sulfatation, amino acid addition such as arginylation or ubiquitination. Such modifications are fully detailed in the literature (Proteins Structure and

Molecular Properties (1993) 2nd Ed., T. E. Creighton, New York; Post-translational Covalent Modifications of Proteins (1983) B. C. Johnson, Ed., Academic Press, New York; Seifter et al. (1990) Analysis for protein modifications and nonprotein cofactors, Meth. Enzymol. 182:626-646 and Rattan et al., (1992) Protein Synthesis: Post-translational Modifications and Aging, Ann NY Acad Sci, 663:48-62). Accordingly, the terms “peptide”, “polypeptide”, “protein” preferably include for example lipopeptides, lipoproteins, glycopeptides, glycoproteins and the like.

Preferably, a (poly) peptide or protein is a “classical” (poly) peptide or protein. A “classical” (poly) peptide or protein is typically composed of amino acids selected from the 20 amino acids defined by the genetic code, linked to each other by a normal peptide bond.

In general, a protein or (poly) peptide may be of any length. In some embodiments, the length of a protein may be assessed in the “mature” protein (in its functional state), i.e. “additional” pre-protein sequences, like signal peptides, which are removed in the mature/functional protein, may not be considered for the length of a protein. Preferably, the length of the polypeptides of the inventions does not exceed 500 amino acids. For example, the maximum length of the polypeptide according to the present invention may be 120 amino acids. In some embodiments, the maximum length of the polypeptide according to the present invention does not exceed 100 amino acids, e.g., not more than 95, 90, 85, 80, 75, 70, 65, 60, 55, 50, 45, 40, 35, 30, 29, 28, 27, 26, 25, 24, 23, 22, 21, or 20 amino acids.

As used herein, the term “microbiota protein” refers to a protein of microbiota origin. The term “microbiota”, as used herein, refers to commensal, symbiotic and pathogenic microorganisms found in and on all multicellular organisms studied to date from plants to animals. In particular, microbiota have been found to be crucial for immunologic, hormonal and metabolic homeostasis of their host. In particular, the microbiota is non-pathogenic. In other words, the microbiota are usually not (capable of) causing a disease in the host and/or they are preferably not harmful to the host. Microbiota include bacteria, archaea, protists, fungi, viruses and phages. Accordingly, a microbiota protein may be a bacterial protein, an archaea protein, a protist protein, a fungi protein, a virus protein and/or a phage protein.

As used herein, the term “recombinant” is intended to refer to such compounds, e.g. proteins, that are prepared, expressed, created or isolated by recombinant means, such as proteins isolated from a heterologous host cell (which does not express said protein in nature) or proteins expressed using a recombinant expression vector transfected into a host cell. Thus, recombinant proteins are proteins that are prepared, expressed, created or isolated by recombinant means, such as proteins isolated from a host cell transfected to express the protein, proteins isolated from a recombinant protein library, and proteins prepared, expressed, created or isolated by any other means that involve splicing of the respective gene sequence encoding the protein into other DNA sequences (distinct from the DNA sequences flanking said gene in nature). In particular, the term “recombinant” is intended to refer to such compounds, e.g. proteins, that do not occur in nature. In some instances, recombinant proteins may have glycosylation patterns, which do not occur in nature.

As well-known in the art, peptides, polypeptides and proteins can be encoded by nucleic acids. The terms “nucleic acid”, “nucleic acid molecule”, “nucleic acid sequence”, “polynucleotide”, “nucleotide sequence” are used herein interchangeable and refer to a precise succession of natural nucleotides (e.g., A, T, G, C and U), or synthetic nucleotides, i.e. to a chain of at least two nucleotides. In particular, the terms “nucleic acid”, “nucleic acid molecule”, “nucleic acid sequence”, “polynucleotide”, “nucleotide sequence” refer to DNA or RNA. Nucleic acids preferably comprise single stranded, double stranded or partially double stranded DNA or RNA, preferably selected from genomic DNA, cDNA, ribosomal

DNA, and the transcription product of said DNA, such as RNA. Preferred examples of nucleic acids include rRNA, mRNA; antisense DNA, antisense RNA; complimentary RNA and/or DNA sequences, ribozyme, (complementary) RNA/DNA sequences with or without expression elements, a vector; a mini-gene, gene fragments, regulatory elements, promoters, and combinations thereof. Further preferred examples of nucleic acid (molecules) and/or polynucleotides include, e.g., a recombinant polynucleotide, a vector, an oligonucleotide, an RNA molecule such as an rRNA, an mRNA, or a tRNA, or a DNA molecule as described above. It is thus preferred that the nucleic acid (molecule) is a DNA molecule or an RNA molecule; preferably selected from genomic DNA; cDNA; rRNA; mRNA; antisense DNA; antisense RNA; complementary RNA and/or DNA sequences; RNA and/or DNA sequences with or without expression elements, regulatory elements, and/or promoters; a vector; and combinations thereof. It is within the skill of the person in the art to determine nucleotide sequences which can encode a specific amino acid sequence.

The polypeptides and/or nucleic acids according to the invention may be prepared by any known method in the art including, but not limited to, any synthetic method, any recombinant method, any ex vivo generation method and the like, and any combination thereof. Such techniques are well-known in the art.

In general, the term “sequence variant”, as used herein, i.e. throughout the present application, refers to a sequence which is similar (meaning in particular at least 50% sequence identity, see below), but not (100%) identical, to a reference sequence (such as any one of the specific sequences described in the sequence listing). Accordingly, a sequence variant contains at least one alteration in comparison to a reference sequence. For example, in a sequence variant one or more of the amino acids or nucleotides of the reference sequence is deleted or substituted, or one or more amino acids or nucleotides are inserted into or added to the sequence of the reference sequence. Therefore, the “sequence variant” is similar, but contains at least one alteration, in comparison to its reference sequence. Preferably, a sequence variant shares, in particular over the whole length of the sequence, at least 70%, preferably at least 75%, more preferably at least 80%, even more preferably at least 85%, still more preferably at least 90%, particularly preferably at least 95% and most preferably at least 98% or 99% sequence identity with a reference sequence. A sequence variant may preserve the specific function of the reference sequence. In the context of the present invention, this function may be the functionality of inducing and/or enhancing IL-10 secretion (from human cells).

The term “sequence variant” includes nucleotide sequence variants and amino acid sequence variants. For example, an amino acid sequence variant has an altered sequence in which one or more of the amino acids is deleted or substituted in comparison to the reference sequence, or one or more amino acids are inserted or added in comparison to the reference amino acid sequence. As a result of the alterations, the amino acid sequence variant has an amino acid sequence which is at least 50%, e.g. at least 70%, preferably at least 75%, more preferably at least 80%, even more preferably at least 85%, still more preferably at least 90%, particularly preferably at least 95% and most preferably at least 98% or 99% identical to the reference sequence. For example, variant sequences which are at least 90% identical have no more than 10 alterations (i.e. any combination of deletions, insertions or substitutions) per 100 amino acids of the reference sequence.

In the context of the present invention, an amino acid sequence “sharing a sequence identity” of at least, for example, 70% to a query (reference) amino acid sequence of the present invention, is intended to mean that the sequence of the subject amino acid sequence is identical to the query sequence except that the subject amino acid sequence may include up to three amino acid alterations per each 10 amino acids of the query amino acid sequence. In other words, to obtain an amino acid sequence having a sequence of at least 70% identity to a query amino acid sequence, up to 30% (3 of 10) of the amino acid residues in the subject sequence may be inserted or substituted with another amino acid or deleted, preferably within the above definitions of variants or fragments. The same, of course, also applies similarly to nucleic acid sequences.

In the context of the present invention, any amino acid substitutions are preferably conservative amino acid substitutions. Examples of conservative substitutions include substitution of one aliphatic residue for another, such as Ile, Val, Leu, or Ala for one another; or substitutions of one polar residue for another, such as between Lys and Arg; Glu and Asp; or Gin and Asn. Other such conservative substitutions, for example, substitutions of entire regions having similar hydrophobicity properties, are well known (Kyte and Doolittle, 1982, J. Mol. Biol. 157 (1): 105-132). Examples of conservative amino acid substitutions are presented in Table 1 below:

TABLE 1

| Original residues | Examples of substitutions |
|---|---|
| Ala (A) | Val, Leu, Ile, Gly |
| Arg (R) | His, Lys |
| Asn (N) | Gln |
| Asp (D) | Glu |
| Cys (C) | Ser |
| Gln (Q) | Asn |
| Glu (E) | Asp |
| Gly (G) | Pro, Ala |
| His (H) | Lys, Arg |
| Ile (I) | Leu, Val, Met, Ala, Phe |
| Leu (L) | Ile, Val, Met, Ala, Phe |
| Lys (K) | Arg, His |
| Met (M) | Leu, Ile, Phe |
| Phe (F) | Leu, Val, Ile, Tyr, Trp, Met |
| Pro (P) | Ala, Gly |
| Ser (S) | Thr |
| Thr (T) | Ser |
| Trp (W) | Tyr, Phe |
| Tyr (Y) | Trp, Phe |
| Val (V) | Ile, Met, Leu, Phe, Ala |

For (amino acid or nucleic acid) sequences without exact correspondence, a “% identity” of a first sequence (e.g., the sequence variant) may be determined with respect to a second sequence (e.g., the reference sequence). In general, the two sequences to be compared may be aligned to give a maximum correlation between the sequences. This may include inserting “gaps” in either one or both sequences, to enhance the degree of alignment. A % identity may then be determined over the whole length of each of the sequences being compared (so-called “global alignment”), that is particularly suitable for sequences of the same or similar length, or over shorter, defined lengths (so-called “local alignment”), that is more suitable for sequences of unequal length.

Methods for comparing the identity (sometimes also referred to as “similarity” or “homology”) of two or more sequences are well known in the art. The percentage to which two (or more) sequences are identical can e.g. be determined using a mathematical algorithm. A preferred, but not limiting, example of a mathematical algorithm which can be used is the algorithm of Karlin et al. (1993), PNAS USA, 90:5873-5877. Such an algorithm is integrated in the BLAST family of programs, e.g. BLAST or NBLAST program (see also Altschul et al., 1990, J. Mol. Biol. 215, 403-410 or Altschul et al. (1997), Nucleic Acids Res, 25:3389-3402), accessible through the home page of the NCBI at world wide web site ncbi.nlm.nih.gov) and FASTA (Pearson (1990), Methods Enzymol. 183, 63-98; Pearson and Lipman (1988), Proc. Natl.

Acad. Sci. U. S. A 85, 2444-2448.). Sequences which are identical to other sequences to a certain extent can be identified by these programmes. Furthermore, programs available in the Wisconsin Sequence Analysis Package, version 9.1 (Devereux et al., 1984, Nucleic Acids Res., 387-395), for example the programs BESTFIT and GAP, may be used to determine the % identity between two polynucleotides and the % identity and the % homology or identity between two polypeptide sequences. BESTFIT uses the “local homology” algorithm of (Smith and Waterman (1981), J. Mol. Biol. 147, 195-197.) and finds the best single region of similarity between two sequences.

By “pharmaceutically acceptable excipient”, it is meant herein a compound of pharmaceutical grade which improves the delivery, stability or bioavailability of an active agent, and can be metabolized by, and is non-toxic to, a subject to whom it is administered. Preferred excipients according to the invention include any of the excipients commonly used in pharmaceutical products, such as, for example, water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable excipients may further comprise minor amounts of auxiliary substances such as wetting or emulsifying agents, or preservatives.

According to the different aspects and embodiments of the invention described herein, a “subject” or “host” preferably refers to a mammal, and most preferably to a human being.

As used herein, the expression “treatment of a disease” generally refers to ameliorating, reducing, preventing and/or treating a disease or condition, or to reducing or preventing the occurrence of a disease or condition or the risk of developing a disease or condition. Accordingly, the expression “treatment of a disease” refers to prophylactic as well as therapeutic settings. Prophylactic settings generally mean to avoid or minimize the onset, development or recurrence of a disease or condition before its onset or after it was “healed”, while therapeutic settings usually encompass reducing, ameliorating or curing a disease or condition (or symptoms of a disease or condition) after its onset. In particular, the term “preventing” encompasses “reducing the likelihood of occurrence of” or “reducing the likelihood of reoccurrence”.

An “effective amount” or “effective dose” as used herein is an amount which provides the desired effect. For therapeutic purposes, an effective amount is an amount sufficient to provide a beneficial or desired clinical result. The preferred effective amount for a given application can be easily determined by the skilled person taking into consideration, for example, the size, age, weight of the subject, the type of disease/disorder to be prevented or treated, and the amount of time since the disease/disorder began. In the context of the present invention, in terms of treatment, an effective amount of the composition is usually an amount that is sufficient to enhance and/or induce IL-10 secretion from human cells.

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded. In the context of the present invention, the term “comprise” encompasses the term “consist of”. The term “comprising” thus encompasses “including” as well as “consisting” e.g., a composition “comprising” X may consist exclusively of X or may include something additional e.g., X+Y.

The terms “a” and “an” and “the” and similar reference used in the context of describing the invention (especially in the context of the claims) are to be construed to cover both the singular and the plural, unless otherwise indicated herein or clearly contradicted by context.

Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention.

The word “substantially” does not exclude “completely” e.g., a composition which is “substantially free” from Y may be completely free from Y. Where necessary, the word “substantially” may be omitted from the definition of the invention.

The term “about” in relation to a numerical value x means x ±10%.

Additional definitions are provided throughout the specification.

The present invention may be understood more readily by reference to the following detailed description, including preferred embodiments of the invention, and examples included herein.

### DETAILED DESCRIPTION

Although the present invention is described in detail below, it is to be understood that this invention is not limited to the particular methodologies, protocols and reagents described herein as these may vary. It is also to be understood that the terminology used herein is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Polypeptides Inducing IL-10 Secretion from Human Cells

In a first aspect the present invention provides a polypeptide comprising or consisting of an amino acid sequence according to general formula (I):

(I)
SCX₁X₂X₃YLX₄

wherein
X₁ may be any amino acid,
X₂ may be any amino acid,
X₃ may be any amino acid or is deleted, and
X₄ is Por D.

Preferably, X₁ is selected from the group consisting of F, A, C, G, I, L, M, P, H, W, Y and V; more preferably X₁ is selected from the group consisting of F, H, W and Y; and particularly preferably X₁ is F.

Preferably, X₂ is selected from the group consisting of F, A, C, G, I, L, M, P, H, W, Y and V; more preferably X₂ is selected from the group consisting of F, H, W and Y; and particularly preferably X₂ is F.

Preferably, X₃ is deleted or selected from the group consisting of I, A, C, G, L, M, F, P, W and V, Y and V; more preferably X₃ is deleted or selected from the group consisting of I, A, G or L; and particularly preferably X₃ is deleted or I.

The present inventors have identified different polypeptides, for example having an amino acid sequence according to SEQ ID NO: 1 or 9, which are capable of inducing and/or enhancing IL-10 secretion from human cells. Based thereon, the inventors identified the sequence motifs described herein, for inducing and/or enhancing IL-10 secretion from human cells.

Preferably, the polypeptide comprises or consists of an amino acid sequence according to general formula (Ia):

(Ia)
KGSRSCX₁X₂X₃YLX₄

wherein X₁-X₄ are defined as described above.

In some embodiments of general formula (I) or (Ia), X₃ may be deleted and X₄ may be P. Accordingly, the polypeptide may comprise or consist of an amino acid sequence according to SEQ ID NO: 3:

(SEQ ID NO: 3)
SCX₁X₂YLP

wherein X₁ and X₂ are as defined above.

Preferably, the polypeptide comprises or consists of an amino acid sequence according to

SEQ ID NO: 5
(SCFFYLP)
or
6
(KKGSRSCFFYLP).

In some embodiments of general formula (I) or (Ia), X₃ may be an amino acid as defined above and X₄ may be D. Accordingly, the polypeptide may comprise or consist of an amino acid sequence according to SEQ ID NO: 4:

(SEQ ID NO: 4)
SCX₁X₂X₃YLD

wherein X₁-X₃ are as defined above.

Preferably, the polypeptide comprises or consists of an amino acid sequence according to

SEQ ID NO: 7
(SCFFIYLD)
or
8
(KGSRSCFFIYLD).

Preferably, the polypeptide comprises two cysteine residues. Without being bound to any theory, the present inventors assume that two cysteine residues may form a disulfide bond, thereby forming the polypeptide into a loop sharing similarities with certain human hormones. Therefore, two cysteine residues may advantageously stabilize the structure of the polypeptide. In some embodiments, no additional cysteines are introduced into polypeptide. It is thus preferred that the polypeptide includes a (single) disulfide bond and/or forms a loop (also referred to as “cyclic” polypeptide). Accordingly, it is preferred that the polypeptide comprises two cysteine residues forming a disulfide bond.

In some embodiments 10 to 20 amino acids (i.e., 10, 11, 12, 13, 14, 15, 16, 17, 18, 19 or 20 amino acids), preferably 11 to 19 amino acids, more preferably 12 to 18 amino acids, even more preferably 13 to 17 amino acids, still more preferably 14 to 16 amino acids, most preferably (exactly) 15 amino acids may be located between the two cysteine residues (and, thus, form the loop between the disulfide bond).

In some embodiments, the polypeptide may comprise two amino acid sequences as described above (e.g., according to general formula (I) and/or (Ia) (or one or more embodiments thereof as described above. Thereby, the polypeptide would comprise two cysteine residues. The two amino acid sequences may be the same or different. For example, the polypeptide may comprise an amino acid sequence according to SEQ ID NO: 5 and an amino acid sequence according to SEQ ID NO: 7; preferably an amino acid sequence according to SEQ ID NO: 6 and an amino acid sequence according to SEQ ID NO: 8.

The present invention also provides a polypeptide comprising or consisting of an amino acid sequence according to general formula (II):

(II)
CX₁X₂X₃YLX₄X₅X₆X₇X₈X₉KGSRX₁₀C

wherein
X₁ may be any amino acid,
X₂ may be any amino acid,
X₃ may be any amino acid or is deleted,
X₄ is P or D,
X₅ may be any amino acid,
X₆ may be any amino acid or is deleted,
X₇ may be any amino acid,
X₈ may be any amino acid,
X₉ is K or Q, and
X₁₀ may be any amino acid.

In particular, X₁-X₄ of general formula (II) are defined as described above for general formula (I) and (Ia). Accordingly, X₁-X₄ of general formula (II) correspond to X₁-X₄ of general formula (I).

Preferably, X₅ is selected from the group consisting of R, N, D, Q, E, H, K, S, T and Y; more preferably X₅ is selected from the group consisting of R, K, Q and H; and particularly preferably X₅ is R.

Preferably, X₆ is deleted or selected from the group consisting of R, N, D, Q, E, H, K, S, T and Y; more preferably X₆ is deleted or selected from the group consisting of R, K, Q and H; and particularly preferably X₆ is deleted or Q.

Preferably, X₇ is selected from the group consisting of G, A, C, I, L, M, S, F, P, W or V; more preferably X₇ is selected from the group consisting of G, A and S; and particularly preferably X₇ is G.

Preferably, X₈ is selected from the group consisting of Y, R, N, D, Q, E, K, H, S and T; more preferably X₈ is selected from the group consisting of Y, T and S; and particularly preferably X₈ is T or Y.

Preferably, X₉ is deleted or selected from the group consisting of R, N, D, Q, E, H, K, S, T and Y; more preferably X₉ is deleted or selected from the group consisting of R, K, Q and H; and particularly preferably X₉ is deleted or K or Q.

Preferably, X₁₀ is selected from the group consisting of G, A, C, I, L, M, S, F, P, W or V; more preferably X₁₀ is selected from the group consisting of G, A and S; and particularly preferably X₁₀ is S or G.

It is also preferred that the polypeptide comprises an amino acid sequence according to general formula (I), e.g. (Ia), and an amino acid sequence according to general formula (II). Thereby, general formulae (I) and (II) may overlap, in particular in (the same) X₁-X₄.

More preferably, the polypeptide comprises or consists of an amino acid sequence according to general formula (IIa):

(IIa)
SCFFX₃YLX₄RX₆GX₈X₉KGSRX₁₀C

wherein
X₃ is I or deleted,
X₄ is P or D,
X₆ is Q or deleted,
X₈ is T or Y,
X₉ is K or Q, and
X₁₀ is S or G.

In the amino acid sequences of general formulae (II) and (IIa), each of X₃ and X₆ may independently be present or absent. For example, both, X₃ or X₆, may be present. Alternative, both, X₃ or X₆, may be deleted. However, it is preferred that either X₃ or X₆ (but not both, X3 and X₆) are deleted. Thereby, 15 amino acid residues are located between the two cysteine residues in general formulae (II) and (IIa).

In some embodiments, the polypeptide of the present invention as described above has a length of at least 17 amino acids, e.g. at least 17, 18, 19, 20, 21, 22, 23, 24, 25, 26, 27 or 28 amino acids. Preferably, the polypeptide of the present invention as described above has a length of at least 20 amino acids. More preferably, the polypeptide of the present invention as described above has a length of at least 25 amino acids, e.g. 28 amino acids.

In a further aspect, the present invention provides a polypeptide comprising or consisting of an amino acid sequence according to SEQ ID NO: 1, wherein, optionally, 0, 1, 2, 3, 4, 5, 6, 7, or 8 amino acids (of SEQ ID NO: 1) may be substituted, added and/or deleted, with the proviso that the serine residue at position 6 of SEQ ID NO: 1 is maintained.

In the following, the amino acid sequence of SEQ ID NO: 1 is shown, wherein the serine residue at position 6 (S6) of SEQ ID NO: 1 is underlined:

KKGSRSCFFIYLDRGTKKGSRSCFFYLP

A polypeptide with the amino acid sequence of SEQ ID NO: 1 is also referred to herein as “NAT_29”.

The present inventors have surprisingly found various microbiota proteins capable of inducing and/or enhancing IL-10 secretion from human cells, as shown in Example 1. As shown in Example 1 (FIG. 1, IL-10 secretion from human cells stimulated with the exemplified microbiota proteins is higher than IL-10 secretion from the same type of human cells stimulated with an E. coli lysate. In particular, the microbiota protein “ID3166” (SEQ ID NO: 2) showed a very high efficacy in inducing and/or enhancing IL-10 secretion even at low doses, as described in Examples 1 and 2. In the following, the amino acid sequence of SEQ ID NO: 2 is shown:

AFLFTSTGVPKKAAEAAFFLYLNKGTKKGSRSCFFIYLDRGT
KKGSRSCFFYLPRQGYQKGSRGCFFIYLDRGTKKGSRGCFFI
YLDCEKRAGNVCIRKCRGRYLHKKTPRRYRNAEATCS

The present inventors focused on the microbiota protein “ID3166” (SEQ ID NO: 2) and identified its fragment of SEQ ID NO: 1 (NAT_29; underlined in the amino acid sequence of SEQ ID NO: 2 shown above), which is capable of inducing and/or enhancing IL-10 secretion from human cells, while it does not stimulate pro-inflammatory cytokines, such as TNF and

IL-6, as shown in the Examples. As shown in the examples, deletion of up to eight amino acids did not abrogate IL-10 secretion. Therefore, polypeptides comprising or consisting of SEQ ID NO: 1, wherein, optionally, 0, 1, 2, 3, 4, 5, 6, 7 or 8 amino acids may be substituted, added and/or deleted, with the proviso that the serine residue at position 6 of SEQ ID NO: 1 is maintained, emerge as candidate drugs with anti-inflammatory properties.

As described above, the amino acid sequence of SEQ ID NO: 1 (“NAT_29”) is a fragment of the microbiota protein according to SEQ ID NO: 2 (“ID3166”). However, the polypeptides of the invention as described herein do preferably not comprise or consist of the amino acid sequence according to SEQ ID NO: 2 (“ID3166”). In some embodiments, the polypeptide of the invention is a recombinant polypeptide (not occurring in nature), e.g. a (recombinant) fusion protein, which may contain the IL-10 secreting polypeptides of the present invention in combination with a different domain/functionality, e.g. a label, targeting or stabilizing moiety (which does not occur in the same protein in nature). Preferably, the polypeptide of the invention does not comprise a (full-length) microbiota protein, e.g. a bacterial or viral protein. In some embodiments, the polypeptide of the invention does not comprise a sequence variant or fragment of a (full-length) microbiota protein, e.g. a bacterial or viral protein, having at least 50%, 55%, 60%, 65%, 70%, 75%, 80%, 85%, 90% or 95% sequence identity to said (full-length) microbiota protein, e.g. to SEQ ID NO: 2.

As described above, the polypeptide may have a (minimum) length of 20 amino acids. In other embodiments, the polypeptide has a (minimum) length of 21 amino acids. In other embodiments, the polypeptide has a (minimum) length of 22 amino acids. In other embodiments, the polypeptide has a (minimum) length of 23 amino acids. In other embodiments, the polypeptide has a (minimum) length of 24 amino acids. In other embodiments, the polypeptide has a (minimum) length of 25 amino acids. In other embodiments, the polypeptide has a (minimum) length of 26 amino acids. In other embodiments, the polypeptide has a (minimum) length of 27 amino acids. Preferably, the polypeptide has a (minimum) length of 28 amino acids.

In some embodiments, the polypeptide has a (maximum) length of 120 amino acids. In other embodiments, the polypeptide has a (maximum) length of 100 amino acids. In other embodiments, the polypeptide has a (maximum) length of 80 amino acids. In other embodiments, the polypeptide has a (maximum) length of 60 amino acids. In other embodiments, the polypeptide has a (maximum) length of 50 amino acids. Preferably, the polypeptide has a (maximum) length of 40 amino acids, e.g. of 35 amino acids.

Accordingly, the polypeptide may have a length of 20-120 amino acids, preferably a length of 22-100 amino acids, more preferably a length of 23-80 amino acids, even more preferably a length of 24-60 amino acids, still more preferably 25-50 amino acids and particularly preferably of 26-40 amino acids, for example 27-35 amino acids.

In some embodiments, the polypeptide comprises or consists of an amino acid sequence according to SEQ ID NO: 1, wherein (exactly) 1, 2, 3, 4, 5, 6, 7 or 8 amino acids amino acids may be substituted, added and/or deleted, with the proviso that the serine residue at position 6 (S6) of SEQ ID NO: 1 is maintained. Accordingly, the polypeptide may comprise (exactly) 1, 2, 3, 4, 5, 6, 7 or 8 amino acids mutations (namely, amino acid substitutions, additions or deletions) compared to SEQ ID NO: 1, as long as the serine at position 6 (S6) of SEQ ID NO: 1 is maintained. Such a sequence variant usually preserves the specific function of the reference sequence, in particular the functionality of inducing and/or enhancing IL-10 secretion (from human cells) as described above.

Preferably, the polypeptide comprises or consists of an amino acid sequence according to SEQ ID NO: 1, wherein (exactly) 1, 2, 3, 4, 5, 6 or 7 amino acids amino acids may be substituted, added and/or deleted, with the proviso that the serine residue at position 6 (S6) of SEQ ID NO: 1 is maintained. More preferably, the polypeptide comprises or consists of an amino acid sequence according to SEQ ID NO: 1, wherein (exactly) 1, 2, 3, 4, 5 or 6 amino acids amino acids may be substituted, added and/or deleted, with the proviso that the serine residue at position 6 (S6) of SEQ ID NO: 1 is maintained. Even more preferably, the polypeptide comprises or consists of an amino acid sequence according to SEQ ID NO: 1, wherein (exactly) 1, 2, 3, 4 or 5 amino acids amino acids may be substituted, added and/or deleted, with the proviso that the serine residue at position 6 (S6) of SEQ ID NO: 1 is maintained. Still more preferably, the polypeptide comprises or consists of an amino acid sequence according to SEQ ID NO: 1, wherein (exactly) 1, 2, 3 or 4 amino acids amino acids may be substituted, added and/or deleted, with the proviso that the serine residue at position 6 (S6) of SEQ ID NO: 1 is maintained. Particularly preferably, the polypeptide comprises or consists of an amino acid sequence according to SEQ ID NO: 1, wherein (exactly) 1, 2 or 3 amino acids amino acids may be substituted, added and/or deleted, with the proviso that the serine residue at position 6 (S6) of SEQ ID NO: 1 is maintained. Most preferably, the polypeptide comprises or consists of an amino acid sequence according to SEQ ID NO: 1, wherein (exactly) 1 or 2 amino acids amino acids may be substituted, added and/or deleted, with the proviso that the serine residue at position 6 (S6) of SEQ ID NO: 1 is maintained.

In some embodiments, the lysine residue at position 1 (K1) of SEQ ID NO: 1 is maintained. In other embodiments, the lysine residue at position 1 (K1) of SEQ ID NO: 1 may be deleted or substituted. While the lysine residue at position 1 (K1) of SEQ ID NO: 1 may be substituted with any amino acid, a conservative substitution is preferred. For example, it may be substituted with another polar amino acid, e.g. selected from the group consisting of R, N, D, Q, E, H, S, T and Y. Preferably, it may be substituted with another basic or positively charged amino acid, e.g. it may be substituted with R or H (arginine or histidine).

In some embodiments, the lysine residue at position 2 (K2) of SEQ ID NO: 1 is maintained. In other embodiments, the lysine residue at position 2 (K2) of SEQ ID NO: 1 may be deleted or substituted. While the lysine residue at position 2 (K2) of SEQ ID NO: 1 may be substituted with any amino acid, a conservative substitution is preferred. For example, it may be substituted with another polar amino acid, e.g. selected from the group consisting of R, N, D, Q, E, H, S, T and Y. Preferably, it may be substituted with another basic or positively charged amino acid, e.g. it may be substituted with R or H (arginine or histidine).

In some embodiments, the glycine residue at position 3 (G3) of SEQ ID NO: 1 is maintained. In other embodiments, the glycine residue at position 3 (G3) of SEQ ID NO: 1 may be deleted or substituted. While the glycine residue at position 3 (G3) of SEQ ID NO: 1 may be substituted with any amino acid, a conservative substitution is preferred. For example, it may be substituted with another nonpolar amino acid, e.g. selected from the group consisting of A, C, I, L, M, F, P, W or V. Preferably, it may be substituted with another aliphatic amino acid, e.g. it may be substituted with A, I or L. It is also preferred that it may be substituted with another very small amino acid, e.g. it may be substituted with A or S (alanine or serine).

In some embodiments, the serine residue at position 4 (S4) of SEQ ID NO: 1 is maintained. In other embodiments, the serine residue at position 4 (S4) of SEQ ID NO: 1 may be deleted or substituted. While the serine residue at position 4 (S4) of SEQ ID NO: 1 may be substituted with any amino acid, a conservative substitution is preferred. For example, it may be substituted with another polar amino acid, e.g. selected from the group consisting of R, N, D, Q, E, K, H, T and Y. Preferably, it may be substituted with another very small amino acid, e.g.

it may be substituted with A or G (alanine or glycine). It is also preferred that it may be substituted with another hydroxylic amino acid, e.g. with T (threonine).

In some embodiments, the arginine residue at position 5 (R5) of SEQ ID NO: 1 is maintained. In other embodiments, the arginine residue at position 5 (R5) of SEQ ID NO: 1 may be deleted or substituted. While the arginine residue at position 5 (R5) of SEQ ID NO: 1 may be substituted with any amino acid, a conservative substitution is preferred. For example, it may be substituted with another polar amino acid, e.g. selected from the group consisting of N, D, Q, E, H, K, S, T and Y. Preferably, it may be substituted with another basic or positively charged amino acid, e.g. it may be substituted with K or H (lysine or histidine).

In some embodiments, the cysteine residue at position 7 (C7) of SEQ ID NO: 1 is maintained. In other embodiments, the cysteine residue at position 7 (C7) of SEQ ID NO: 1 may be deleted or substituted. While the cysteine residue at position 7 (C7) of SEQ ID NO: 1 may be substituted with any amino acid, a conservative substitution is preferred. For example, it may be substituted with another nonpolar amino acid, e.g. selected from the group consisting of A, G, I, L, M, F, P, W or V. Preferably, it may be substituted with another small amino acid, e.g. it may be substituted with A, G or S, preferably with A or G (alanine or glycine).

In some embodiments, the phenylalanine residue at position 8 (F8) of SEQ ID NO: 1 is maintained. In other embodiments, the phenylalanine residue at position 8 (F8) of SEQ ID NO: 1 may be deleted or substituted. While the phenylalanine residue at position 8 (F8) of

SEQ ID NO: 1 may be substituted with any amino acid, a conservative substitution is preferred. For example, it may be substituted with another nonpolar amino acid, e.g. selected from the group consisting of A, C, G, I, L, M, P, W or V. Preferably, it may be substituted with another aromatic amino acid, e.g. it may be substituted with H, W or Y (histidine, tryptophan or tyrosine).

In some embodiments, the phenylalanine residue at position 9 (F9) of SEQ ID NO: 1 is maintained. In other embodiments, the phenylalanine residue at position 9 (F9) of SEQ ID NO: 1 may be deleted or substituted. While the phenylalanine residue at position 9 (F9) of SEQ ID NO: 1 may be substituted with any amino acid, a conservative substitution is preferred. For example, it may be substituted with another nonpolar amino acid, e.g. selected from the group consisting of A, C, G, I, L, M, P, W or V. Preferably, it may be substituted with another aromatic amino acid, e.g. it may be substituted with H, W or Y (histidine, tryptophan or tyrosine).

In some embodiments, the isoleucine residue at position 10 (110) of SEQ ID NO: 1 is maintained. In other embodiments, the isoleucine residue at position 10 (110) of SEQ ID NO: 1 may be deleted or substituted. While the isoleucine residue at position 10 (110) of SEQ ID NO: 1 may be substituted with any amino acid, a conservative substitution is preferred. For example, it may be substituted with another nonpolar amino acid, e.g. selected from the group consisting of A, C, G, L, M, F, P, W or V. Preferably, it may be substituted with another aliphatic amino acid, e.g. it may be substituted with A, G or L (alanine, glycine or leucine).

In some embodiments, the tyrosine residue at position 11 (Y11) of SEQ ID NO: 1 is maintained. In other embodiments, the tyrosine residue at position 11 (Y11) of SEQ ID NO: 1 may be deleted or substituted. While the tyrosine residue at position 11 (Y11) of SEQ ID NO: 1 may be substituted with any amino acid, a conservative substitution is preferred. For example, it may be substituted with another polar amino acid, e.g. selected from the group consisting of R, N, D, Q, E, K, H, S and T. Preferably, it may be substituted with another aromatic amino acid, e.g. it may be substituted with H, W or F (histidine, tryptophan or phenylalanine).

In some embodiments, the leucine residue at position 12 (L12) of SEQ ID NO: 1 is maintained. In other embodiments, the leucine residue at position 12 (L12) of SEQ ID NO: 1 may be deleted or substituted. While the leucine residue at position 12 (L12) of SEQ ID NO: 1 may be substituted with any amino acid, a conservative substitution is preferred. For example, it may be substituted with another nonpolar amino acid, e.g. selected from the group consisting of A, C, G, I, M, F, P, W or V. Preferably, it may be substituted with another aliphatic amino acid, e.g. it may be substituted with A, G or I (alanine, glycine or isoleucine).

In some embodiments, the aspartic acid residue at position 13 (D13) of SEQ ID NO: 1 is maintained. In other embodiments, the aspartic acid residue at position 13 (D13) of SEQ ID NO: 1 may be deleted or substituted. While the aspartic acid residue at position 13 (D13) of SEQ ID NO: 1 may be substituted with any amino acid, a conservative substitution is preferred. For example, it may be substituted with another polar amino acid, e.g. selected from the group consisting of R, N, Q, E, K, H, S, T and Y. Preferably, it may be substituted with another negatively charged or acidic amino acid, such as E (glutamic acid). It is also preferred that D13 is substituted with P (proline).

In some embodiments, the arginine residue at position 14 (R14) of SEQ ID NO: 1 is maintained. In other embodiments, the arginine residue at position 14 (R14) of SEQ ID NO: 1 may be deleted or substituted. While the arginine residue at position 14 (R14) of SEQ ID NO: 1 may be substituted with any amino acid, a conservative substitution is preferred. For example, it may be substituted with another polar amino acid, e.g. selected from the group consisting of N, D, Q, E, H, K, S, T and Y. Preferably, it may be substituted with another basic or positively charged amino acid, e.g. it may be substituted with K or H (lysine or histidine).

In some embodiments, the glycine residue at position 15 (G15) of SEQ ID NO: 1 is maintained. In other embodiments, the glycine residue at position 15 (G15) of SEQ ID NO: 1 may be deleted or substituted. While the glycine residue at position 15 (G15) of SEQ ID NO: 1 may be substituted with any amino acid, a conservative substitution is preferred. For example, it may be substituted with another nonpolar amino acid, e.g. selected from the group consisting of A, C, I, L, M, F, P, W or V. Preferably, it may be substituted with another aliphatic amino acid, e.g. it may be substituted with A, I or L. It is also preferred that it may be substituted with another very small amino acid, e.g. it may be substituted with A or S (alanine or serine).

In some embodiments, the threonine residue at position 16 (T16) of SEQ ID NO: 1 is maintained. In other embodiments, the threonine residue at position 16 (T16) of SEQ ID NO: 1 may be deleted or substituted. While the threonine residue at position 16 (T16) of SEQ ID NO: 1 may be substituted with any amino acid, a conservative substitution is preferred. For example, it may be substituted with another polar amino acid, e.g. selected from the group consisting of R, N, D, Q, E, K, H, S and Y. Preferably, it may be substituted with A, S, Y or G (alanine, serine, tyrosine or glycine), in particular with the S or Y (serine or tyrosine).

In some embodiments, the lysine residue at position 17 (K17) of SEQ ID NO: 1 is maintained. In other embodiments, the lysine residue at position 17 (K17) of SEQ ID NO: 1 may be deleted or substituted. While the lysine residue at position 17 (K17) of SEQ ID NO: 1 may be substituted with any amino acid, a conservative substitution is preferred. For example, it may be substituted with another polar amino acid, e.g. selected from the group consisting of R, N, D, Q, E, H, S, T and Y. Preferably, it may be substituted with Q, R or H (glutamine, arginine or histidine).

In some embodiments, the lysine residue at position 18 (K18) of SEQ ID NO: 1 is maintained. In other embodiments, the lysine residue at position 18 (K18) of SEQ ID NO: 1 may be deleted or substituted. While the lysine residue at position 18 (K18) of SEQ ID NO: 1 may be substituted with any amino acid, a conservative substitution is preferred. For example, it may be substituted with another polar amino acid, e.g. selected from the group consisting of R, N, D, Q, E, H, S, T and Y. Preferably, it may be substituted with another basic or positively charged amino acid, e.g. it may be substituted with R or H (arginine or histidine).

In some embodiments, the glycine residue at position 19 (G19) of SEQ ID NO: 1 is maintained. In other embodiments, the glycine residue at position 19 (G19) of SEQ ID NO: 1 may be deleted or substituted. While the glycine residue at position 19 (G19) of SEQ ID NO: 1 may be substituted with any amino acid, a conservative substitution is preferred. For example, it may be substituted with another nonpolar amino acid, e.g. selected from the group consisting of A, C, I, L, M, F, P, W or V. Preferably, it may be substituted with another aliphatic amino acid, e.g. it may be substituted with A, I or L. It is also preferred that it may be substituted with another very small amino acid, e.g. it may be substituted with A or S (alanine or serine).

In some embodiments, the serine residue at position 20 (S20) of SEQ ID NO: 1 is maintained. In other embodiments, the serine residue at position 20 (S20) of SEQ ID NO: 1 may be deleted or substituted. While the serine residue at position 20 (S20) of SEQ ID NO: 1 may be substituted with any amino acid, a conservative substitution is preferred. For example, it may be substituted with another polar amino acid, e.g. selected from the group consisting of R, N, D, Q, E, K, H, T and Y. Preferably, it may be substituted with another very small amino acid, e.g. it may be substituted with A or G (alanine or glycine). It is also preferred that it may be substituted with another hydroxylic amino acid, e.g. with T (threonine).

In some embodiments, the arginine residue at position 21 (R21) of SEQ ID NO: 1 is maintained. In other embodiments, the arginine residue at position 21 (R21) of SEQ ID NO: 1 may be deleted or substituted. While the arginine residue at position 21 (R21) of SEQ ID NO: 1 may be substituted with any amino acid, a conservative substitution is preferred. For example, it may be substituted with another polar amino acid, e.g. selected from the group consisting of N, D, Q, E, H, K, S, T and Y. Preferably, it may be substituted with another basic or positively charged amino acid, e.g. it may be substituted with K or H (lysine or histidine).

In some embodiments, the serine residue at position 22 (S22) of SEQ ID NO: 1 is maintained. In other embodiments, the serine residue at position 22 (S22) of SEQ ID NO: 1 may be deleted or substituted. While the serine residue at position 22 (S22) of SEQ ID NO: 1 may be substituted with any amino acid, a conservative substitution is preferred. For example, it may be substituted with another polar amino acid, e.g. selected from the group consisting of R, N, D, Q, E, K, H, T and Y. Preferably, it may be substituted with another very small amino acid, e.g. it may be substituted with A or G (alanine or glycine). It is also preferred that it may be substituted with another hydroxylic amino acid, e.g. with T (threonine).

In some embodiments, the cysteine residue at position 23 (C23) of SEQ ID NO: 1 is maintained. In other embodiments, the cysteine residue at position 23 (C23) of SEQ ID NO: 1 may be deleted or substituted. While the cysteine residue at position 23 (C23) of SEQ ID NO: 1 may be substituted with any amino acid, a conservative substitution is preferred. For example, it may be substituted with another nonpolar amino acid, e.g. selected from the group consisting of A, G, I, L, M, F, P, W or V. Preferably, it may be substituted with another small amino acid, e.g. it may be substituted with A, G or S, preferably with A or G (alanine or glycine).

In some embodiments, the phenylalanine residue at position 24 (F24) of SEQ ID NO: 1 is maintained. In other embodiments, the phenylalanine residue at position 24 (F24) of SEQ ID NO: 1 may be deleted or substituted. While the phenylalanine residue at position 24 (F24) of

SEQ ID NO: 1 may be substituted with any amino acid, a conservative substitution is preferred. For example, it may be substituted with another nonpolar amino acid, e.g. selected from the group consisting of A, C, G, I, L, M, P, W or V. Preferably, it may be substituted with another aromatic amino acid, e.g. it may be substituted with H, W or Y (histidine, tryptophan or tyrosine).

In some embodiments, the phenylalanine residue at position 25 (F25) of SEQ ID NO: 1 is maintained. In other embodiments, the phenylalanine residue at position 25 (F25) of SEQ ID NO: 1 may be deleted or substituted. While the phenylalanine residue at position 25 (F25) of SEQ ID NO: 1 may be substituted with any amino acid, a conservative substitution is preferred. For example, it may be substituted with another nonpolar amino acid, e.g. selected from the group consisting of A, C, G, I, L, M, P, W or V. Preferably, it may be substituted with another aromatic amino acid, e.g. it may be substituted with H, W or Y (histidine, tryptophan or tyrosine).

In some embodiments, the tyrosine residue at position 26 (Y26) of SEQ ID NO: 1 is maintained. In other embodiments, the tyrosine residue at position 26 (Y26) of SEQ ID NO: 1 may be deleted or substituted. While the tyrosine residue at position 26 (Y26) of SEQ ID NO: 1 may be substituted with any amino acid, a conservative substitution is preferred. For example, it may be substituted with another polar amino acid, e.g. selected from the group consisting of R, N, D, Q, E, K, H, S and T. Preferably, it may be substituted with another aromatic amino acid, e.g. it may be substituted with H, W or F (histidine, tryptophan or phenylalanine).

In some embodiments, the leucine residue at position 27 (L27) of SEQ ID NO: 1 is maintained. In other embodiments, the leucine residue at position 27 (L27) of SEQ ID NO: 1 may be deleted or substituted. While the leucine residue at position 27 (L27) of SEQ ID NO: 1 may be substituted with any amino acid, a conservative substitution is preferred. For example, it may be substituted with another nonpolar amino acid, e.g. selected from the group consisting of A, C, G, I, M, F, P, W or V. Preferably, it may be substituted with another aliphatic amino acid, e.g. it may be substituted with A, G or I (alanine, glycine or isoleucine).

In some embodiments, the proline residue at position 28 (P28) of SEQ ID NO: 1 is maintained. In other embodiments, the proline residue at position 28 (P28) of SEQ ID NO: 1 may be deleted or substituted. While the proline residue at position 28 (P28) of SEQ ID NO: 1 may be substituted with any amino acid, a conservative substitution is preferred. For example, it may be substituted with another nonpolar amino acid, e.g. selected from the group consisting of A, C, G, I, L, M, F, W or V. Preferably, it may be substituted with another small amino acid, e.g. it may be substituted with A, G, S or T (alanine, glycine, serine or threonine).

Preferably, the cysteine residue(s) at position 7 and/or 23 (C7 and/or C23) of SEQ ID NO: 1 is/are maintained in the polypeptide of the present invention (in addition to the serine residue at position 6 (S6) of SEQ ID NO: 1). As described above, without being bound to any theory, the present inventors assume that the cysteine residues at positions 7 and 23 may form a disulfide bond, thereby forming the polypeptide into a loop sharing similarities with certain human hormones. Therefore, the cysteine at positions 7 and 23 of SEQ ID NO: 1 may advantageously stabilize the structure of the polypeptide. In some embodiments, no additional cysteines (in addition to C7 and C23) are introduced into SEQ ID NO: 1. It is thus preferred that the polypeptide includes a (single) disulfide bond and/or forms a loop (also referred to as “cyclic” polypeptide). In some embodiments 10 to 20 amino acids (i.e., 10, 11, 12, 13, 14, 15, 16, 17, 18, 19 or 20 amino acids), preferably 11 to 19 amino acids, more preferably 12 to 18 amino acids, even more preferably 13 to 17 amino acids, still more preferably 14 to 16 amino acids, most preferably (exactly) 15 amino acids may be located between the two cysteine residues (and, thus, form the loop between the disulfide bond).

It is also preferred that—in addition to the serine residue at position 6 (S6) of SEQ ID NO: 1—also the serine residue at position 22 (S22) of SEQ ID NO: 1 is maintained. More preferably, the serine residues at positions 6 (S6) and 22 (S22) of SEQ ID NO: 1 as well as the cysteine residues at position 7 (C7) and 23 (C23) of SEQ ID NO: 1 are maintained; while 0, 1, 2, 3, 4 or 5 of the remaining amino acids of SEQ ID NO: 1 (i.e., of the amino acids other than S6, C7, S22 and C23) may be substituted, added and/or deleted.

In some embodiments, the polypeptide comprises the sequence motif YLP and/or YLD (e.g., corresponding to positions 11-13 and/or 26-28 of SEQ ID NO: 1). For example, Y11, L12 and D13 in SEQ ID NO: 1 may be maintained. Alternatively or additionally, Y26, L27 and P28 of SEQ ID NO: 1 may be maintained. Preferably, the sequence motif YLP or YLD is located downstream (on the C-terminal side) of a cysteine residue, e.g. at a position corresponding to C7 and/or C23 of SEQ ID NO: 1. Thereby, two or three amino acids may be located between the cysteine residue and the YLP or YLD motif, e.g. as shown in SEQ ID NOs 3 and 4 below (wherein X may be any amino acid):

(SEQ ID NO: 3)
SCXXYLP
(SEQ ID NO: 4)
SCXXXYLD

Preferably, the amino acids located between the cysteine residue and the YLP or YLD motif is a phenylalanine residue or an isoleucine residue, e.g. as shown in SEQ ID NOs 5 and 7 below (wherein X may be any amino acid):

(SEQ ID NO: 5)
SCFFYLP
(SEQ ID NO: 7)
SCFFIYLD

Preferably, the amino acid sequence according to SEQ ID NOs 6 and/or 8 may be maintained in SEQ ID NO: 1:

(SEQ ID NO: 6)
KKGSRSCFFYLP
(SEQ ID NO: 8)
KKGSRSCFFIYLD

In some embodiments, the polypeptide of the present invention may comprise or consist of an amino acid sequence according to SEQ ID NO: 1 (NAT_29).

In some embodiments, the polypeptide of the present invention may comprise or consist of an amino acid sequence according to SEQ ID NO: 9 (NAT_38).

In particular, the polypeptides of the present invention are capable of inducing and/or enhancing IL-10 secretion from human cells. In view of the functionality to induce and/or enhance IL-10 secretion from human cells, the polypeptide of the present invention may be also referred to as “secretagogue”. In general, the term “secretagogue” refers to substances causing other substances to be secreted.

Various human cells were described to secrete IL-10, including immune cells and non-immune cells (e.g., epithelial and neuronal cells). Preferably, the polypeptide induces and/or enhances IL-10 secretion from human immune cells, e.g. peripheral blood mononuclear cells (PBMCs). For example, various blood cell types, including lymphocytes, monocytes, dendritic cells and granulocytes were reported to produce IL-10. In some embodiments, the polypeptide induces and/or enhances IL-10 secretion from monocytes. In some embodiments, the polypeptide induces and/or enhances IL-10 secretion from lymphocytes, e.g. T lymphocytes.

The skilled person is aware of various methods to identify whether a polypeptide is capable of inducing and/or enhancing IL-10 secretion from human cells. For example, human cells capable of IL-10 secretion (as outlined above, e.g. PBMCs, lymphocytes, monocytes, dendritic cells or granulocytes) may be cultured in presence and absence of the polypeptide and the IL-10 secretion in both cases (presence/absence of the polypeptide) may be compared. It is understood that for such a comparative purpose, the human cells used as well as the other experimental conditions (except for the presence/absence of the polypeptide) are usually identical. In some embodiments, for assessing induction of IL-10 secretion, the culture medium may be devoid of IL-10 inducing compounds (other than the polypeptide, for the respective experimental group). In other embodiments, for assessing enhancement of IL-10 secretion, an IL-10 inducing compound (other than the polypeptide) may be added to observe whether or not IL-10 secretion can be increased. Examples of IL-10 inducing compounds useful for such experiments include lipopolysaccharide (LPS), lysates from bacteria, phytohemagglutinin (PHA) and other compounds known to induce inflammatory responses or to induce IL-10 directly (e.g., in humans/human cells). In comparative cells cultured in absence of the polypeptide of the invention, a control compound may be used. For example, such cells may be cultured in the presence of a negative control (not IL-10 inducing/enhancing) or of positive control (known to be IL-10 inducing/enhancing). Kits for determining IL-10 secretion are commercially available, e.g. Human IL-10 AlphaLisa® kit (Perkin), “IL-10 Human ELISA Kit” (Invitrogen); “Human IL-10 Quantikine ELISA Kit D1000B” (R&D Systems); “Simoa® IL-10 Advantage Kit” (Quanterix); “IL-10 Secretion Assay” (Miltenyi Biotec); “Human IL10 kit” (Cisbio); “Human IL-10 ELISA Kit” (Abcam) etc. A specific example for determining whether a polypeptide is capable of inducing and/or enhancing IL-10 secretion from human cells, is provided in the example section of the present specification.

In general, a polypeptide is considered to be capable of inducing and/or enhancing IL-10 secretion from human cells, if human cells increase more IL-10, when cultured in the presence of said polypeptide as compared to the same human cell type cultured under the same conditions, but in the absence of said polypeptide.

In some embodiments, secretion of IL-10 from human cells cultured in the presence of the polypeptide of the invention is at least 1.5-fold, 2-fold, 2.5-fold, 3-fold, 4-fold, 5-fold, 6-fold, 7-fold, 8-fold, 9-fold or 10-fold higher than the IL-10 level produced by the same type of human cells cultured in the absence of the polypeptide of the invention and/or cultured with a control compound.

In some embodiments, IL-10 secretion from human cells stimulated with the polypeptide of the present invention is the same or higher than IL-10 secretion from the same type of human cells stimulated with LPS, e.g. with 10 to 100 ng/ml, such as 10, 20, 30, 40, 50, 60, 70, 80 90 or 100 ng/ml LPS (but otherwise under the same conditions). In some embodiments, IL-10 secretion from human cells stimulated with said polypeptide is the same or higher than IL-10 secretion stimulated with 10 or 100 ng/ml lipopolysaccharide (LPS).

In some embodiments, IL-10 secretion from human cells stimulated with the polypeptide of the present invention is higher than IL-10 secretion from the same type of human cells stimulated with an E. coli lysate. A (cell-free) E. coli lysate is commercially available or may be prepared by methods known in the art, e.g. by sonication, by homogenization, by enzymatic lysis (e.g., using lysosyme) or by freezing and grinding. Preferably, the E. coli lysate may be prepared according to the method described in Zubay G. In vitro synthesis of protein in microbial systems. Annu Rev Genet. 1973;7:267-87, which is incorporated herein by reference. Further suitable methods to prepare high yield E. coli lysate are described in Kim, D M., Swartz, J. R. Oxalate improves protein synthesis by enhancing ATP supply in a cell-free system derived from Escherichia coli. Biotechnology Letters 22, 1537-1542 (2000). https://doi.org/10.1023/A: 1005624811710; and in Kim D M, Swartz J R. Regeneration of adenosine triphosphate from glycolytic intermediates for cell-free protein synthesis. Biotechnol Bioeng. 2001 Aug. 20;74 (4): 309-16; which are incorporated herein by reference. An E. coli strain with low exonuclease activity may be used and growth conditions may be optimized to allow optimum protein expression from linear and plasmid templates.

In some embodiments, IL-10 secretion from human cells stimulated with the polypeptide of the present invention is higher than IL-10 secretion from the same type of human cells stimulated with phytohemagglutinin (PHA). Phytohemagglutinin (PHA) is an extract from the red kidney bean (Phaseolus vulgaris) that contains potent cell-agglutinating and mitogenic properties. PHA is known to induce IL-10 secretion from human cells and, thus, it is often used as positive control. In certain embodiments, IL-10 secretion from human cells stimulated with 0.1 μM of the polypeptide of the present invention is higher than IL-10 secretion from the same type of human cells stimulated with 10 μg/ml PHA. In certain instances, IL-10 secretion from human cells stimulated with concentrations as low as 0.025 μM of the polypeptide of the present invention is still higher than IL-10 secretion from the same type of human cells stimulated with 10 μg/ml PHA.

### Production of Polypeptides

The polypeptide of the invention may be prepared by artificial, recombinant or synthetic means (i.e., the polypeptide may be prepared either through recombinant protein synthesis or by means of chemical peptide synthesis). For example, the polypeptide may be prepared by chemical synthesis, in vitro or expressed by another organism (other than the organism from which the sequence is obtained; “heterologous expression”). Accordingly, the polypeptide of the invention is in particular a recombinant polypeptide. In some embodiments, the polypeptide of the invention is prepared by chemical synthesis. In some embodiments, the polypeptide of the invention is prepared by in vitro synthesis (cell-free expression) or by recombinant overexpression (e.g. in bacteria cells). Preferably, the polypeptide is purified.

The skilled person is aware of various methods to prepare polypeptides based on the amino acid sequence or the nucleic acid sequence. For example, the polypeptide may be prepared by chemical synthesis, in vitro synthesis (cell-free expression) or by (in vivo) recombinant (over) expression.

For example, the polypeptide of the invention may be expressed (heterologously) by bacteria, e.g. E. coli, transformed with an expression vector comprising a nucleic acid sequence encoding the polypeptide as well-known in the art.

In vitro protein synthesis (also referred to as “in vitro protein expression”, “cell-free protein expression” and “cell-free protein synthesis”) is the production of recombinant proteins in solution using biomolecular translation machinery extracted from cells. In vitro protein synthesis occurs in cell lysates or in cocktails of recombinant proteins rather than within cultured cells and, thus, it is achieved without the use of living cells. The in vitro protein synthesis environment is not constrained by a cell wall or homeostasis conditions necessary to maintain cell viability. Accordingly, in vitro protein synthesis enables direct access and control of the translation environment. Moreover, this technique enables rapid expression and manufacture of functional proteins. In vitro protein synthesis is useful for various applications including optimization of protein production, optimization of protein complexes, to study protein synthesis, incorporating non-natural amino acids, high-throughput screens, functional analyses, molecular interaction detection, molecular structure and localization analyses and molecular diagnostics.

Common components of a cell-free reaction comprise proteins necessary to achieve in vitro transcription and transduction, such as cocktails of recombinant proteins or cell extracts. In addition, an energy source, a supply of amino acids, a nucleic acid encoding the polypeptide to be expressed and, optionally, a cofactor, such as magnesium, may be added. In vitro protein synthesis can be accomplished with several kinds and species of cell extracts or with cocktails of recombinant proteins. A cell extract may be obtained, for example, by lysing a cell of interest and centrifuging out the cell walls, DNA genome, and other debris, such that the necessary cell machinery (including ribosomes, aminoacyl-tRNA synthetases, translation initiation and elongation factors, nucleases, etc.) remains. Examples include cell extracts made from E. coli (ECE), rabbit reticulocytes (RRL), wheat germ (WGE), insect cells (ICE, for example SF9 or SF21) and human cells, which are all commercially available. Examples of commercially available in vitro protein synthesis systems include, but are not limited to, RTS (5 PRIME); Expressway™ (Life Technologies); S30 T7 high yield (Promega); One-step human IVT (Thermo Scientific); WEPRO® (CellFree Sciences); TNT® coupled (Promega); RTS CECF (5 PRIME); TNT® Coupled (Promega); Retic lysate IVT™ (Life Technologies); TNT® T7 (Promega); EasyXpress Insect kit (Qiagen/RiN A); PURExpress® (New England Biolabs); and PURESYSTEM® (BioComber)). In some embodiments, PURExpress® (New England Biolabs) may be used, which is a cocktail of recombinant proteins necessary to achieve in vitro transcription and transduction. In other embodiments, bacterial extracts or lysates, e.g. from E. coli, in particular ECE (E. coli extract), may be used, since they provide the bacterial machinery (which is ideal for expression of bacterial proteins) and typically achieve high yields. A more detailed description of in vitro protein synthesis in bacterial extracts may be derived from Hani S. Zaher, Rachel Green: Chapter One-In Vitro Synthesis of Proteins in Bacterial Extracts, Editor(s): Jon Lorsch, Methods in Enzymology, Academic Press, Vol. 539, 2014, p. 3-15, ISSN 0076-6879, ISBN 9780124201200. Kits for cell-free heterologous expression are commercially available, e.g. Escherichia coli Cell Free kit (RTS 100 E. coli Disulfide Kit; Biotechrabbit, Hennigsdorf, Germany).

The nucleic acid used in in vitro protein synthesis is preferably RNA or DNA. For example, isolated RNA (in particular mRNA) synthesized in vivo or in vitro may be used as template for translation. It is also preferred to use DNA, in particular a coupled translation/transcription system, in which circular or linear DNA, such as a gene/an ORF cloned into a plasmid vector (cDNA), or a linear DNA template, such as a PCR-generated template, are used. The nucleic acid may be codon optimized. In some embodiments, direct synthesis of DNA is used, preferably with codon optimization.

The nucleic acid, e.g., a synthetic DNA molecule, may be subcloned into a vector or a plasmid, e.g. for (over) expression or heterologous expression of the encoded protein. The vector may be an expression vector, which may be used for production of expression products such as polypeptides. For example, an expression vector or a plasmid for expression may comprise sequences needed for transcription of a sequence stretch of the vector, such as a promoter sequence (e.g., a T7 promoter). Accordingly, the vector may comprise a (T7) promoter. A “promoter” is usually a DNA sequence that directs the transcription of a polynucleotide encoding the polypeptide (of the invention). A promoter is usually located in the 5′ region of a gene, in particular proximal to the transcriptional start site of polynucleotide encoding the polypeptide (of the invention). A promoter may be inducible. In addition, the vector may contain a particular tag, if required. In some embodiments, the vector or plasmid contains regulatory elements or control elements for heterologous expression of the polypeptide of the invention in bacterial cells.

In vitro/cell-free protein synthesis is preferably applied to proteins/polypeptides having a minimum length of 20 amino acids, preferably having a minimum length of 30 amino acids, more preferably having a minimum length of 40 amino acids, even more preferably having a minimum length of 45 amino acids and still more preferably having a minimum length of 50 amino acids. For example, polypeptide having a length (preferably without signal peptide) of 50 to 350 amino acids or 50 to 500 amino acids are synthesized by in vitro/cell-free protein synthesis as described above.

Alternatively, the polypeptide of the invention may be prepared by chemical synthesis, as known in the art and described, for example, by Fields GB. Introduction to peptide synthesis. Curr Protoc Protein Sci. 2002; Chapter 18: Unit-18.1. doi: 10.1002/0471140864.ps1801s26. For example, solid phase technologies known in the art (solid-phase peptide synthesis (SPPS)) may be used, e.g. applying Fmoc-based chemistries. SPPS allows the rapid assembly of a peptide chain through successive reactions of amino acid derivatives on an insoluble porous support. Alternatively, liquid-phase peptide synthesis (LPPS) may be used. Various commercial suppliers of chemical protein and peptide synthesis are available, e.g. Pepscan (Lelystad, Netherlands), GenScript (Piscataway, NJ, USA), LifeTein (Somerset, NJ, USA), JPT (Berlin, Germany), SB-Peptide (Saint Egrève, France).

Chemical synthesis, e.g. SPPS, may be applied to polypeptides having a maximum length of 100 amino acids, preferably having a maximum length of 90 amino acids, more preferably having a maximum length of 80 amino acids, even more preferably having a maximum length of 70 or 60 amino acids and still more preferably having a maximum length of 50 amino acids. For example, polypeptides having a length of 15 to 50 amino acids or 20 to 50 amino acids are synthesized by chemical synthesis, e.g. SPPS.

In some embodiments, longer polypeptides, e.g. having a length as described above (e.g., 50 -350 amino acids), may be synthesized by in vitro/cell-free protein synthesis or recombinant overexpression and shorter proteins, e.g. having a length as described above (e.g., 20-50 amino acids) may be synthesized by chemical synthesis, e.g. SPPS.

### Nucleic Acids and Vectors Encoding the Polypeptide

In a further aspect, the present invention also provides a nucleic acid comprising a polynucleotide encoding the polypeptide according to the present invention as described above.

A nucleic acid (molecule) is a molecule comprising nucleic acid components. The term nucleic acid (molecule) usually refers to DNA or RNA (molecules). It may be used synonymous with the term “polynucleotide”, i.e. the nucleic acid molecule may consist of a polynucleotide encoding the polypeptide of the invention. Alternatively, the nucleic acid molecule may also comprise further elements in addition to the polynucleotide encoding the polypeptide. Typically, a nucleic acid molecule is a polymer comprising or consisting of nucleotide monomers which are covalently linked to each other by phosphodiester-bonds of a sugar/phosphate-backbone. The term “nucleic acid (molecule)” also encompasses modified nucleic acid (molecules), such as base-modified, sugar-modified or backbone-modified etc. DNA or RNA molecules.

Nucleic acids preferably comprise single stranded, double stranded or partially double stranded nucleic acids, preferably selected from genomic DNA, cDNA, RNA, SiRNA, antisense DNA, antisense RNA, ribozyme, complimentary RNA/DNA sequences with or without expression elements, a mini-gene, gene fragments, regulatory elements, promoters, and combinations thereof. Further preferred examples of nucleic acid (molecules) include, e.g., a recombinant polynucleotide, a vector, an oligonucleotide, an RNA molecule such as an rRNA, an mRNA or a tRNA, or a DNA molecule as described above. It is thus preferred that the nucleic acid (molecule) is a DNA molecule or an RNA molecule; preferably selected from genomic DNA; cDNA; rRNA; mRNA; antisense DNA; antisense RNA; complimentary

RNA and/or DNA sequences; RNA and/or DNA sequences with or without expression elements, regulatory elements, and/or promoters; a vector; and combinations thereof.

Nucleic acids encoding polypeptides according to the invention may be in the form of naked nucleic acids, or nucleic acids cloned into plasmids or viral vectors (Tregoning and Kinnear, Using Plasmids as DNA Vaccines for Infectious Diseases. Microbiol Spectr. 2014 December;2 (6). doi: 10.1128/microbiolspec.PLAS-0028-2014), the latter being preferred. Examples of suitable viral vectors according to the invention include, without limitation, retrovirus, adenovirus, adeno-associated virus (AAV), herpes virus and poxvirus vectors. It is within the skill of the person in the art to clone a nucleic acid into a plasmid or viral vector, using standard recombinant techniques in the art.

In general, the nucleic acid (molecule) may be manipulated to insert, delete or alter certain nucleic acid sequences. Changes from such manipulation include, but are not limited to, changes to introduce restriction sites, to amend codon usage, to add or optimize transcription and/or translation regulatory sequences, etc. It is also possible to change the nucleic acid to alter the encoded amino acids. For example, it may be useful to introduce one or more (e.g., 1, 2, 3, 4, 5, 6, 7, 8, 9, 10, etc.) amino acid substitutions, deletions and/or insertions into the polypeptide's amino acid sequence. Such point mutations can modify effector functions, post-translational modifications, immunogenicity, etc., can introduce amino acids for the attachment of covalent groups (e.g., labels) or can introduce tags (e.g., for purification purposes). Alternatively, a mutation in a nucleic acid sequence may be “silent”, i.e. not reflected in the amino acid sequence due to the redundancy of the genetic code. In general, mutations can be introduced in specific sites or can be introduced at random, followed by selection (e.g., molecular evolution). For instance, one or more nucleic acids encoding the polypeptide of the invention can be randomly or directionally mutated to introduce different properties in the encoded amino acids. Such changes can be the result of an iterative process wherein initial changes are retained and new changes at other nucleotide positions are introduced. Further, changes achieved in independent steps may be combined.

In some embodiments, the polynucleotide encoding the polypeptide (or the (complete) nucleic acid molecule) may be codon-optimized. The skilled artisan is aware of various tools for codon optimization, such as those described in: Ju Xin Chin, Bevan Kai-Sheng Chung, Dong-Yup Lee, Codon Optimization OnLine (COOL): a web-based multi-objective optimization platform for synthetic gene design, Bioinformatics, Volume 30, Issue 15, 1 Aug. 2014, Pages 2210-2212; or in: Grote A, Hiller K, Scheer M, Munch R, Nortemann B, Hempel DC, Jahn D, JCat: a novel tool to adapt codon usage of a target gene to its potential expression host. Nucleic Acids Res. 2005 Jul. 1;33 (Web Server issue): W526-31; or in US 2011/0081708 A1; or as provided by commercial suppliers, e.g., the codon optimization algorithm provided by Twist Bioscience (San Francisco, USA). In some embodiments, the polynucleotide encoding the polypeptide (or the (complete) nucleic acid molecule) is codon-optimized for expression by prokaryotic cells, preferably it is codon-optimized for expression in bacteria, such as E. coli.

In particular, preferred embodiments of the polypeptide according to the present invention as described above also apply for such a nucleic acid according to the present invention. For example, the nucleic acid may encode the polypeptide comprising or consisting of an amino acid sequence as set forth in SEQ ID NO: 1, wherein, optionally, 0, 1, 2, 3, 4, 5, 6, 7 or 8 amino acids may be substituted, added and/or deleted, with the proviso that the serine residue at position 6 of SEQ ID NO: 1 is maintained.

The nucleotide sequence of SEQ ID NO: 10:

AAAAAAGGCAGCAGAAGCTGCTTTTTTATTTACCTCGACAGG
GGTACCAAAAAAGGCAGCAGAAGCTGCTTTTTTTATTTACCT

is a specific example for a nucleotide sequence encoding the amino acid sequence of SEQ ID NO: 1.

Accordingly, it is preferred that the polynucleotide encoding the polypeptide of the invention comprises or consists of a nucleic acid sequence as set forth in SEQ ID NO: 10 or of a sequence variant thereof as described above. For example, the nucleic acid sequence of said polynucleotide may share at least 80% sequence identity with SEQ ID NO: 10. It is understood that for any sequence variant, a nucleotide sequence encoding the serine at position 6 (S6) of SEQ ID NO: 1 is maintained. More preferably, the polynucleotide encoding the polypeptide has a nucleic acid sequence according to SEQ ID NO: 10. However, it is understood that due to the redundancy of the genetic code, distinct nucleic acid sequences may encode the same amino acid sequence. Accordingly, the polypeptide of SEQ ID NO: 1 can also be encoded by nucleic acids distinct from the above-mentioned exemplified nucleic acids.

Due to the redundancy of the genetic code, the present invention also comprises sequence variants of nucleic acid sequences, which encode the same amino acid sequences. The polynucleotide encoding the polypeptide may be optimized for expression of the polypeptide. For example, codon optimization of the nucleotide sequence may be used to improve the efficiency of translation in expression systems for the production of the polypeptide. Moreover, the nucleic acid molecule may comprise heterologous elements (i.e., elements, which in nature do not occur on the same nucleic acid molecule as the coding sequence for the polypeptide). For example, a nucleic acid molecule may comprise a heterologous promotor, a heterologous enhancer, a heterologous UTR (e.g., for optimal translation/expression), a heterologous Poly-A-tail, and the like.

In some embodiments, the nucleic acid may further comprise (in addition to the polynucleotide encoding the polypeptide according to the invention) a polynucleotide encoding a signal peptide (a secretion peptide or export peptide) or a secretion tag.

The signal peptide or secretion tag typically indicates that the protein is destined for the secretory pathway. Accordingly, proteins comprising a signal peptide or a secretion tag are typically secreted from the cell, e.g. from a bacterium expressing said proteins. Signal peptides are usually located at the N-terminus of the protein (i.e., N-terminal). They are typically recognized by the secretion machinery of the cell. Non-limiting examples of signal peptides suitable for protein secretion are described in Degering et al. (Degering C et al., Optimization of protease secretion in Bacillus subtilis and Bacillus licheniformis by screening of homologous and heterologous signal peptides. Appl Environ Microbiol. 2010 October;76 (19): 6370-6. doi: 10.1128/AEM.01146-10) and Watanabe et al. (Watanabe K et al., Scanning the Corynebacterium glutamicum R genome for high-efficiency secretion signal sequences. Microbiology (Reading). 2009 March; 155 (Pt 3): 741-750. doi: 10.1099/mic.0.024075-0.), which are incorporated herein by reference in its entirety.

In bacteria, two major export pathways, the general secretion or Sec pathway and the twin-arginine translocation or Tat pathway, exist for the transport of proteins across the plasma membrane as described in Freudl R. Signal peptides for recombinant protein secretion in bacterial expression systems. Microb Cell Fact. 2018 Mar. 29;17 (1): 52. In some embodiments, the polynucleotide encodes a secretion tag, which is a prototypical N-terminal Sec-dependent secretion signal or Tat-dependent secretion signal.

The polynucleotide encoding the signal peptide and the polynucleotide encoding the polypeptide according to the invention, may be comprised in the same open reading frame (ORF). Accordingly, after expression, the polypeptide of the invention may be operably linked to the signal peptide. In other words, protein expression may yield a fusion protein comprising (i) the signal peptide, and (ii) the polypeptide according to the invention. Thereby, the signal peptide is preferably located N-terminal of the polypeptide according to the invention; more preferably, the signal peptide is located at the N-terminus (of the fusion protein). Accordingly, the polynucleotide encoding the signal peptide and the polynucleotide encoding the polypeptide according to the invention, may be arranged such that the polynucleotide encoding the signal peptide is located at the 5′ end of the sequence encoding the fusion protein.

In some embodiments, the signal peptide or secretion tag may be removed (e.g. by cleavage), e.g. before, during or after secretion, such that the (mature) protein does not comprise the signal peptide. In particular, the signal peptide or secretion tag may be cleaved upon secretion into the periplasmic space. For example, the secretion system may be able to remove the signal peptide before secreting the protein from the host cell, e.g. the engineered bacteria. For example, in Type V auto-secretion-mediated secretion the N-terminal signal peptide is removed upon translocation of the “passenger” peptide from the cytoplasm into the periplasmic compartment by the native secretion system. Further, once the auto-secretor is translocated across the outer membrane the C-terminal secretion tag can be removed by either an autocatalytic or protease-catalyzed (e.g., OmpT) cleavage thereby releasing the polypeptide of the invention into the extracellular milieu.

The fusion protein comprising the signal peptide and the polypeptide according to the invention may be a recombinant fusion protein. Accordingly, the signal peptide and the polypeptide according to the invention, may not occur (together) in nature. For example, the signal peptide and/or the polypeptide according to the invention may be recombinant (not occur in nature) or-if both occur in nature-they may occur in nature in distinct organisms, leading to “heterologous” secretion of the protein. Accordingly, the nucleic acid may be recombinant, e.g. comprising a polynucleotide encoding the recombinant fusion protein (which does not occur in nature), and/or a codon-optimized nucleic acid.

In a further aspect, the present invention also provides an expression cassette comprising the polynucleotide encoding the polypeptide of the invention; or the nucleic acid (molecule) of the invention as described above. In addition, an expression cassette usually comprises-operably linked to said polynucleotide or nucleic acid encoding the polypeptide-a regulatory element for expression of the polypeptide of the invention. In some embodiments, the expression cassette comprises a regulatory element for recombinant expression, such as heterologous expression and/or overexpression, of the encoded polypeptide.

As described above, “heterologous expression” means that the encoded polypeptide is expressed by another organism (other than the organism naturally encoding and/or expressing the polypeptide). In other words, “heterologous expression” refers to expression of an encoded protein in a (host) organism, which does not naturally encode (or express) said protein. Recombinant “overexpression” means that more copies of the protein are produced as compared to natural expression. For example, if a bacterium normally expresses a protein, it may be transformed, e.g. with a plasmid or vector for overexpression (e.g., using a particular promoter to increase expression), such that the transformed (genetically engineered) bacterium thereafter expresses more copies of the protein.

The regulatory element may be a (heterologous) promoter. A heterologous promoter does not occur in nature in combination with the respective coding sequence (i.e., the coding sequence is naturally under the control of a distinct promoter). The promoter initiates the transcription and is therefore the point of control for the expression of the encoded protein. The promoter may be inducible, such that protein synthesis is only initiated when required by the introduction of an inducer, such as IPTG. However, gene expression however may also be constitutive (i.e. the protein may be constantly expressed). Examples of promoters include the promoter of the lac operon or the T7 promoter. They may be regulated by the lac operator. A promoter may also be a hybrid of different promoters, for example, the Tac-Promoter, which is a hybrid of trp and lac promoters.

For example, the expression cassette may comprise (operably linked to each other): a (heterologous) promoter, an open reading frame (ORF) comprising or consisting of the polynucleotide encoding the polypeptide of the invention, and a 3′ untranslated region (3′UTR). In some embodiments, the expression cassette comprises (operably linked to each other): a (heterologous) promoter, a ribosomal binding site, the polynucleotide encoding the polypeptide of the invention, and a transcription terminator (for terminating DNA transcription). In some embodiments, the expression cassette may comprise (operably linked to each other): a (heterologous) promoter and an open reading frame (ORF) comprising or consisting of the polynucleotide encoding the signal peptide or secretion tag as described above, and the polynucleotide encoding the polypeptide of the invention. In some embodiments, the expression cassette comprises (operably linked to each other): a (heterologous) promoter; a ribosomal binding site; an ORF comprising or consisting of the polynucleotide encoding the signal peptide or secretion tag as described above, and the polynucleotide encoding the polypeptide of the invention; and a transcription terminator (for terminating DNA transcription).

An expression cassette typically comprises the gene to be expressed (in the present case the polynucleotide/nucleic acid encoding the polypeptide of the invention and a regulatory element for expression. An expression cassette may be a component of a vector, in particular of an expression vector, such that the encoded protein can be expressed by a cell comprising said vector, e.g. a cell transfected with said vector. The expression cassette usually directs the cell's machinery to produce RNA and protein(s). In some embodiments, the expression cassette may be designed for modular cloning of protein-encoding sequences, such that the same cassette can easily be altered to make different proteins. The regulatory element of the expression cassette is usually heterologous in comparison to the encoded protein (to be expressed), i.e. the specific combination of regulatory element and polynucleotide sequence encoding the protein of interest does typically not occur in nature. The regulatory element usually controls the expression of the encoded protein.

The expression cassette may further comprise a nucleic acid sequence encoding a tag (for example, directly downstream of the polynucleotide encoding the polypeptide according to the present invention), such that the expressed polypeptide contains a tag. A tag may be useful, for example, for purification of the polypeptide after expression or as reporters (labels). Examples of tags include histidine (His) tags, other marker peptides, or fusion partners such as glutathione S-transferase or maltose-binding protein.

The expression cassette may be optimized for expression in eukaryotic or prokaryotic cells. For example, an expression cassette for prokaryotes expression vectors may include a Shine-Dalgarno sequence at its translation initiation site for the binding of ribosomes, while an expression cassette for eukaryotic expression may contain the Kozak consensus sequence. Preferably, the expression cassette is optimized for expression in a prokaryotic cell, such as a bacterium.

Further included within the scope of the invention are vectors, for example, expression vectors, comprising the nucleic acid according to the present invention as described above or the expression cassette according to the present invention as described above. For example, the nucleic acid molecule as described above may be a vector. Accordingly, the present invention also provides a vector, comprising the nucleic acid according to the present invention as described above or the expression cassette according to the present invention as described above.

A vector is usually a (recombinant) nucleic acid molecule, which does not occur in nature. Accordingly, the vector may comprise heterologous elements (i.e., sequence elements of different origin in nature). For example, the vector may comprise a multi cloning site, a heterologous promotor, a heterologous enhancer, a heterologous selection marker (to identify cells comprising said vector in comparison to cells not comprising said vector) or combinations thereof. A vector in the context of the present invention is suitable for incorporating or harboring a desired nucleic acid sequence. Such vectors may be storage vectors, expression vectors, cloning vectors, transfer vectors etc. A storage vector is a vector which allows the convenient storage of a nucleic acid molecule. Thus, the vector may comprise a sequence corresponding to (or encoding), e.g., the polypeptide according to the present invention. An expression vector may be used for production of expression products such as RNA, e.g. mRNA, or peptides, polypeptides or proteins. For example, an expression vector may comprise sequences needed for transcription of a sequence stretch of the vector, such as a (heterologous) promoter sequence, e.g. as described above. A cloning vector is typically a vector that contains a cloning site, which may be used to incorporate nucleic acid sequences into the vector. A cloning vector may be, e.g., a plasmid vector or a bacteriophage vector. A transfer vector may be a vector which is suitable for transferring nucleic acid molecules into cells or organisms, for example, viral vectors.

A vector in the context of the present invention may be, e.g., an RNA vector or a DNA vector. Preferably, a vector is a DNA molecule. For example, a vector in the sense of the present application comprises a cloning site, a selection marker, such as an antibiotic resistance factor, and a sequence suitable for multiplication of the vector, such as an origin of replication. Preferably, a vector in the context of the present application is a plasmid vector. Preferably, a vector in the context of the present application is an expression vector. An expression vector may further comprise a nucleic acid sequence encoding a tag (for example, directly downstream of the polynucleotide encoding the polypeptide according to the present invention), such that the expressed polypeptide contains a tag. A tag may be useful, for example, for purification of the protein after expression or as reporters (labels). Examples of tags include histidine (His) tags, other marker peptides, or fusion partners such as glutathione

S-transferase or maltose-binding protein. Furthermore, the expression vector may comprise a polynucleotide encoding a signal peptide or a secretion tag, as described above (e.g. for expression of a fusion protein comprising the signal peptide and the polypeptide of the invention as described above).

A preferred vector is a vector for expression in bacterial cells, such as E. coli. Many expression vectors for expression of a polypeptide/protein of interest in bacterial cells, such as E. coli, are commercially available.

The vector may be useful for expression in so-called “live bacterial vectors”, wherein live bacterial cells (such as bacteria or bacterial spores, e.g., endospores, exospores or microbial cysts) can be administered. Preferred examples thereof are described in Pálffy R, Gardlík R, Hodosy J, Behuliak M, Resko P, Radvánský J, Celec P. Bacteria in gene therapy: bactofection versus alternative gene therapy. Gene Ther. 2006 January; 13 (2): 101-5.

### Cells and Culture Medium

In a further aspect, the present invention also provides a (host) cell expressing the polypeptide according to the invention; or comprising the nucleic acid, the expression cassette or the vector according to the present invention. The (host) cell may be an isolated cell.

Examples of such cells include but are not limited to, eukaryotic cells, e.g., yeast cells, animal cells or plant cells or prokaryotic cells, including E. coli. In some embodiments, the cells are mammalian cells, such as a mammalian cell line. Examples include human cells, CHO cells, HEK293T cells, PER.C6 cells, NSO cells, human liver cells, myeloma cells or hybridoma cells.

Preferably, the (host) cell is a bacterial cell, such as an E. coli cell. Such a cell is preferably used for production of the polypeptide according to the present invention. Moreover, such the (host) cell may also be an active component in a pharmaceutical composition.

Preferably, the (host) cell is a bacterial cell, more preferably a gut bacterial cell. Such a bacterial cell may serve as “live bacterial vector”, wherein live bacterial cells (such as bacteria or bacterial spores, e.g., endospores, exospores or microbial cysts) can serve as vectors to provide the polypeptide of the invention or a nucleic acid encoding the same. Preferred examples thereof are described in Pálffy R, Gardlík R, Hodosy J, Behuliak M, Resko P, Radvánský J, Celec P. Bacteria in gene therapy: bactofection versus alternative gene therapy. Gene Ther. 2006 January;13 (2): 101-5. Bacterial cells (such as bacteria or bacterial spores, e.g., endospores, exospores or microbial cysts), in particular (entire) gut bacterial species, can be advantageous, as they have the potential to trigger a greater immune response than the proteins or nucleic acids they contain.

Moreover, bacterial cells, in particular gut bacteria, according to the invention may be provided in the form of probiotics, i.e. of live gut bacterium, which can thus be used as food additive due to the health benefits it can provide. Those can be for example lyophilized in granules, pills or capsules, or directly mixed with dairy products for consumption.

Accordingly, the present invention also provides a genetically engineered bacterium (or a recombinant bacterium) capable of inducing and/or enhancing IL-10 secretion from a human cell, the bacterium comprising the nucleic acid according to the present invention, the expression cassette according to the present invention, or the vector according to the present invention as described above. Furthermore, the present invention also provides a genetically engineered bacterium (or a recombinant bacterium) (over) expressing the polypeptide according to the present invention. In particular, the genetically engineered bacterium (or recombinant bacterium) is capable of secreting the polypeptide according to the present invention. Accordingly, the present invention also provides a genetically engineered bacterium (or a recombinant bacterium) capable of secreting the polypeptide according to the present invention. To this end, the genetically engineered bacterium (or recombinant bacterium) may comprise an expression cassette as described above. In some embodiments, the bacterium comprises an expression vector (or plasmid) as described above. In some embodiments, the expression cassette may be integrated into the genome of the bacterium, in particular for recombinant expression (e.g., heterologous expression or overexpression).

Preferably, the bacterial (host) cell/bacterium is an engineered bacterium (or a recombinant bacterium). In general, an engineered bacterium (or a recombinant bacterium) is a bacterium, which does not occur in nature. In particular, the genetically engineered bacterium (or recombinant bacterium) may express the polypeptide in a recombinant manner, e.g. by heterologous expression or overexpression. To this end, the (host) bacterium may be modified, e.g. by introducing a vector (or plasmid) encoding the polypeptide into the (host) bacterium (e.g., by transformation). Said vector, or a portion thereof, which includes the polynucleotide encoding the polypeptide, may optionally integrate into the genome of the (host) bacterium.

In some embodiments, a bacterial (host) cell may be genetically modified for better penetration through the cellular membrane; for the facilitation of the release of the carried plasmid or expressed polypeptide; or to reduce the risk of clinically symptomatic infections to a minimum. Engineered bacteria are described, for example, in Pálffy R, Gardlík R, Hodosy J, Behuliak M, Resko P, Radvánský J, Celec P. Bacteria in gene therapy: bactofection versus alternative gene therapy. Gene Ther. 2006 January;13 (2): 101-5. Engineered bacteria secreting proteins of interest are described, for example, in WO 2016/164636, in WO 2018/045184 or in WO 2020/206221 A1, which are incorporated herein by reference.

In some embodiments, the genetically engineered bacterium (or recombinant bacterium) may comprise a polynucleotide encoding a signal peptide or a secretion tag as described above. Accordingly, the genetically engineered bacterium (or recombinant bacterium) may comprise a secretion system, in particular for secreting the polypeptide of the invention. As used herein, the term “secretion system” refers to a native or non-native secretion mechanism capable of secreting or exporting the polypeptide of the invention from the bacterial cytoplasm. The secretion system may comprise a single protein or may comprise two or more proteins assembled in a complex, as known in the art. Non-limiting examples of secretion systems for gram negative bacteria include the modified type III flagellar, type I (e.g., hemolysin secretion system), type Il, type IV, type V, type VI, and type VII secretion systems, resistance-nodulation-division (RND) multi-drug efflux pumps, various single membrane secretion systems. Non-liming examples of secretion systems for gram positive bacteria include Sec and TAT secretion systems.

In general, the (engineered) bacterium is preferably a non-pathogenic bacterium. The term “non-pathogenic bacterium”, as used herein, refers to bacteria that are essentially not capable of causing disease or harmful responses in a host. In some embodiments, non-pathogenic bacteria are commensal bacteria. Examples of non-pathogenic bacteria include, but are not limited to Bacillus, Bacteroides, Bifidobacterium, Brevibacteria, Clostridium, Enterococcus, Escherichia coli (E. coli), Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii. Among those bacteria, (engineered) E. coli is particularly preferred.

In a further aspect, the present invention also provides a culture medium comprising the polypeptide according to the invention, the (host) cell according to the invention, or the bacterium according to the invention.

The culture medium is preferably a culture medium for human cells, in particular human immune cells. Various culture media for human immune cells, such as PBMCs, are known in the art and commercially available. Examples include RPMI-1640 medium, Iscove's Modified Dulbecco's Medium (IMDM), TexMACS medium, and AIM V Medium (ThermoFisher). The culture medium may be supplemented, e.g. with antibiotics, glutamine and/or (inactivated) fetal bovine serum (FBS). In some embodiments, the culture medium is serum-free. Preferably, the culture medium does not contain IL-10 and/or compounds inducing IL-10 secretion from human cells other than the polypeptide according to the invention, the (host) cell according to the invention, or the bacterium according to the invention.

In some embodiments, the culture medium further comprises an antigen, e.g. a self-antigen. For instance, in allergy and autoimmunity, patient-derived immune cells (such as dendritic cells) pulsed with a specific antigen (Ag) can be used to induce differentiation of autologous Ag-specific T regulatory type 1 (Tr1) cell products and, therefore, promote/restore Ag-specific tolerance.

In a further aspect, the present invention also provides an isolated human cell cultured with the culture medium as described above, i.e. comprising the polypeptide according to the invention, the (host) cell according to the invention, or the bacterium according to the invention. The polypeptide according to the invention (or cells expressing such polypeptides) is usually capable of inducing and/or enhancing IL-10 secretion from human cells as described above. Preferably, the human cell cultured with the polypeptide (or cells expressing such polypeptides) is a human immune cell, e.g. a peripheral blood mononuclear cell (PBMC). For example, various blood cell types, including lymphocytes, monocytes, dendritic cells and granulocytes were reported to produce IL-10. In some embodiments, the human cell cultured with the polypeptide of the invention (or cells expressing such polypeptides) is a lymphocyte, e.g. a T lymphocyte.

As described above, culturing human cells (as described above) in the presence of the polypeptide of the invention (or cells expressing such polypeptides) usually results in inducing and/or enhancing IL-10 release from the human cells. Further details regarding Il-10 secretion of human cells in the presence of the polypeptide of the invention are provided above, which apply here accordingly.

### Pharmaceutical Compositions and Medical Treatment and Uses

In a further aspect, the present invention provides a pharmaceutical composition comprising the polypeptide of the invention as described above;
the nucleic acid as described above;
the vector as described above;
the (host) cell as described above;
the bacterium as described above; or
the human cell as described above.

Preferably, the pharmaceutical composition further comprises a pharmaceutically acceptable excipient, diluent or carrier. Although the carrier or excipient may facilitate administration, it should not itself induce the production of antibodies harmful to the individual receiving the composition. Nor should it be toxic. Suitable carriers may be large, slowly metabolized macromolecules such as proteins, polypeptides, liposomes, polysaccharides, polylactic acids, polyglycolic acids, polymeric amino acids, amino acid copolymers and inactive virus particles. In some embodiments, the pharmaceutically acceptable carrier, diluent and/or excipient in the pharmaceutical composition according to the present invention is not an active component in respect to inducing and/or enhancing IL-10 secretion from human cells.

Pharmaceutically acceptable salts can be used, for example mineral acid salts, such as hydrochlorides, hydrobromides, phosphates and sulphates, or salts of organic acids, such as acetates, propionates, malonates and benzoates.

Pharmaceutically acceptable carriers in a pharmaceutical composition may additionally contain liquids such as water, saline, glycerol and ethanol. Additionally, auxiliary substances, such as wetting or emulsifying agents or pH buffering substances, may be present in such compositions. Such carriers enable the pharmaceutical compositions to be formulated as tablets, pills, dragees, capsules, liquids, gels, syrups, slurries and suspensions, for ingestion by the subject. A thorough discussion of pharmaceutically acceptable carriers is available in Gennaro (2000) Remington: The Science and Practice of Pharmacy, 20th edition, ISBN: 0683306472.

Pharmaceutical compositions of the invention may have a pH between 5.5 and 8.5, in some embodiments this may be between 6 and 8, for example about 7. The pH may be maintained by the use of a buffer. The composition may be sterile and/or pyrogen free. The composition may be isotonic with respect to humans. In some embodiments pharmaceutical compositions of the invention are supplied in hermetically-sealed containers.

In some embodiments, the (only) active ingredient in the composition is the polypeptide, the nucleic acid, the vector, the (host) cell, the bacterium or the human cell according to the present invention as described above. As such, it may be susceptible to degradation in the gastrointestinal tract. Thus, if the composition is to be administered by a route using the gastrointestinal tract, the composition may contain agents which protect the said active component from degradation.

In some embodiments, the polypeptide of the invention may be administered in the form of a micro-organism such as a (gut) bacterial cell. Moreover, (gut) bacteria according to the invention may be in the form of probiotics, i.e. of a live bacterium, which can thus be used as food additive due to the health benefits it can provide. Those can be for example lyophilized in granules, pills or capsules, or directly mixed with dairy products for consumption.

The polypeptide of the invention, the nucleic acid (or vector) and the compositions according to the invention can further be encapsulated so as to facilitate their administration to the subject in need thereof. For example, they may be encapsulated into peptide nanocarriers, into virosomes, or into lipid-based carrier systems such as liposome-polycation-DNA complex (Trovato M, De Berardinis P. Novel antigen delivery systems. World J Virol. 2015 Aug. 12;4 (3): 156-68; Saade F, Petrovsky N. Technologies for enhanced efficacy of DNA vaccines. Expert Rev Vaccines. 2012 February; 11 (2): 189-209; Li et al., Peptide Vaccine: Progress and Challenges. Vaccines (Basel). 2014 Jul. 2;2 (3): 515-36).

The composition according to the invention can further comprise other active agents, for example such, which can enhance the effects of the polypeptide of the invention. Alternatively, the composition may not comprise any other active agents (i.e., other than the polypeptide according to the present invention, the cell according to the present invention, the nucleic acid according to the present invention, or the host cell according to the present invention.

In view of the IL-10 secretion induced and/or enhanced by the polypeptide according to the present invention, the present invention also provides a method for inducing and/or enhancing IL-10 secretion in a subject, comprising the step of administering to said subject the polypeptide according to the present invention, the nucleic acid according to the present invention, the vector according to the present invention, the (host) cell according to the present invention, the bacterium according to the present invention.

Preferably, the polypeptide according to the present invention, the nucleic acid according to the present invention, the vector according to the present invention, the (host) cell according to the present invention, the bacterium according to the present invention, or the human immune cell according to the present invention may be used as a medicament.

In other words, the present invention also provides the polypeptide according to the present invention, the nucleic acid according to the present invention, the vector according to the present invention, the (host) cell according to the present invention, the bacterium according to the present invention, the human immune cell according to the present invention, or the pharmaceutical composition according to the present invention for use in medicine.

IL-10 is often considered as the “prototype of anti-inflammatory cytokines” and its inhibitory action is exerted mainly against the most typical markers of inflammation such as IL-1, IL-6, TNF-α, GM-CSF and TNF-γ. The three major pro-inflammatory cytokines IL-1β, IL-6, and TNF-α are responsible for the onset and the maintenance of the inflammatory state in many diseases. Moreover, a TH1 immune response is known to be a characteristic of autoimmune and inflammatory diseases. While IL-10 is able to inhibit both the Th1-type and the Th2-type responses, the effect on Th1 subpopulation is predominant and IL-10 is considered as a promoter of the Th2 response (pleiotropic effect). Accordingly, inhibition/reduction of pro-inflammatory cytokines and Th1 immune responses by inducing or enhancing secretion of IL-10 from human cells is helpful in various inflammatory and autoimmune diseases.

In some embodiments, the polypeptide according to the present invention, the nucleic acid according to the present invention, the vector according to the present invention, the (host) cell according to the present invention, the bacterium according to the present invention, or the human immune cell according to the present invention are thus useful for increasing the secretion of anti-inflammatory cytokines such as IL-10.

Moreover, the Examples of the present application show increased intestinal epithelial cell barrier function upon application of the polypeptide of the invention in assays assessing the transepithelial electrical resistance. Accordingly, the polypeptide according to the present invention, the nucleic acid according to the present invention, the vector according to the present invention, the (host) cell according to the present invention, the bacterium according to the present invention, or the human immune cell according to the present invention may thus be useful for increasing intestinal epithelial cell barrier function or integrity (in a subject). They can therefore be used in vivo for increasing intestinal epithelial cell wound healing or for reducing the intestinal tissue pathology, specifically the gastrointestinal mucosa inflammation in a subject in need thereof, e.g., in a subject having intestinal tissue damages due to a treatment with a chemical. Structural and functional integrity of an epithelial cell layer may be assessed, for example, with a TER assay, which is well-known in the art and described, for example, in: Srinivasan et al., 2015, J Lab Autom, 20:107-126 as well as in Example 6 below.

Accordingly, the polypeptide according to the present invention, the nucleic acid according to the present invention, the vector according to the present invention, the (host) cell according to the present invention, the bacterium according to the present invention, the human immune cell according to the invention, or the pharmaceutical composition according to the present invention may be useful in various medical applications including treatment of inflammatory diseases, autoimmune disorders, and diseases of the gastrointestinal tract (GIT), such as inflammatory bowel diseases (IBD). Inflammatory bowel diseases (IBD) include, for example, Crohn's disease (CD) and ulcerative colitis (UC). Accordingly, the polypeptide according to the present invention, the nucleic acid according to the present invention, the vector according to the present invention, the (host) cell according to the present invention, the bacterium according to the present invention, the human immune cell according to the invention, or the pharmaceutical composition according to the present invention may be used for treating an inflammatory bowel disease (IBD).

In general, inflammatory diseases include a vast array of disorders and conditions that are characterized by inflammation. Inflammatory diseases may be acute or chronic. Examples of inflammatory diseases include allergy such as food allergy, asthma such as allergic asthma, chronic obstructive pulmonary disease (COPD), inflammatory bowel disease (IBD) such as Crohn's disease (CD) and ulcerative colitis (UC), psoriasis, atopic dermatitis (AD), rheumatoid arthritis (RA), lupus such as systemic lupus erythematosus (SLE), multiple sclerosis and Type 1 diabetes (T1D).

In some embodiments, the polypeptide according to the present invention, the nucleic acid according to the present invention, the vector according to the present invention, the (host) cell according to the present invention, the bacterium according to the present invention, the human immune cell according to the invention, or the pharmaceutical composition according to the present invention may be used for treating an allergy such as food allergy.

Moreover, the polypeptide according to the present invention, the nucleic acid according to the present invention, the vector according to the present invention, the (host) cell according to the present invention, the bacterium according to the present invention, the human immune cell according to the invention, or the pharmaceutical composition according to the present invention may be used for any one (or combinations) of:
reducing gastrointestinal inflammation in a patient in need thereof,
reducing intestinal mucosal inflammation in a patient in need thereof,
increasing gastrointestinal wound healing in a patient in need thereof,
increasing intestinal epithelial cell proliferation in a patient in need thereof, and/or
treating or preventing epithelial barrier function disorders, in a patient in need thereof.

Said epithelial barrier function disorder can be for example chosen in the group consisting of: inflammatory bowel disease (IBD), ulcerative colitis (UC), pediatric UC, Crohn's disease (CD), pediatric Crohn's disease, short bowel syndrome, mucositis GI mucositis, oral mucositis, mucositis of the esophagus, stomach, small intestine (duodenum, jejunum, ileum), large intestine (colon), and/or rectum, chemotherapy-induced mucositis, radiation-induced mucositis, necrotizing enterocolitis, pouchitis, a metabolic disease, celiac disease, irritable bowel syndrome (IBS), or chemotherapy associated steatohepatitis (CASH).

Preferably, said epithelial barrier function disorder is an IBD, such as Crohn's Disease (CD). Another preferred disorder to be treated is ulcerative colitis (UC).

Accordingly, the present invention provides a method for reducing, treating, alleviating symptoms of or ameliorating an inflammatory disease or an autoimmune disorder in a subject, comprising the step of administering to said subject the polypeptide according to the present invention, the nucleic acid according to the present invention, the vector according to the present invention, the (host) cell according to the present invention, the bacterium according to the present invention, the human immune cell according to the present invention or the pharmaceutical composition according to the present invention.

Moreover, the present invention provides a method for reducing, treating, alleviating symptoms of or ameliorating an allergy in a subject, comprising the step of administering to said subject the polypeptide according to the present invention, the nucleic acid according to the present invention, the vector according to the present invention, the (host) cell according to the present invention, the bacterium according to the present invention, the human immune cell according to the present invention or the pharmaceutical composition according to the present invention.

The present invention also provides a method for inducing tolerance in a subject, comprising the step of administering to said subject the polypeptide according to the present invention, the nucleic acid according to the present invention, the vector according to the present invention, the (host) cell according to the present invention, the bacterium according to the present invention, or the human immune cell according to the present invention.

Methods of administration of the polypeptide according to the present invention, the nucleic acid according to the present invention, the vector according to the present invention, the (host) cell according to the present invention, the bacterium according to the present invention, or the human immune cell according to the present invention are well-known to the person skilled in the art. For example, it may be directly administered into the subject, into the affected organ (i.e. local administration) or systemically (i.e. enteral or parenteral administration). Enteral administrations include oral and rectal administrations, as well as administrations via gastric feeding tubes, duodenal feeding tubes or gastrostomy. Parenteral administrations include, among others, subcutaneous, intravenous, intramuscular, intra-arterial, intradermal, intraosseous, intracerebral, and intrathecal injections. The administration method will often depend upon the type of disease to be treated and on the type of active compound. For example, the administration is preferably via an enteral route, in particular for the treatment of diseases of the gastrointestinal tract (GIT), such as inflammatory bowel diseases (IBD). In some embodiments, the administration is preferably an oral administration, e.g. if the polypeptide is delivered in the form of a (gut) bacterium as defined above, e.g. if the gut bacterium is in the form of probiotics.

### BRIEF DESCRIPTION OF THE FIGURES

In the following a brief description of the appended figures will be given. The figures are intended to illustrate the present invention in more detail. However, they are not intended to limit the subject matter of the invention in any way.

FIG. 1 shows for Example 1 the results of AlphaLISA for IL-10 secretion from human (CD14 negative) PBMCs by stimulation with 10 exemplified microbiota proteins in the second round of cell-free synthesis.

FIG. 2: shows for Example 2 the results of AlphaLISA for IL-10 secretion from human PBMCs by stimulation with 5 selected microbiota proteins at doses of 0.5, 0.25, 0.1 and 0.025 μM (final concentration).

FIG. 3: shows for Example 3 the NAT_29 dose response on monocyte-derived dendritic cells (MoDCs; A) and monocytes (B). Cells were co-stimulated with NAT_29 (SEQ ID NO: 1; at concentrations of 1000-100-10-1-0.1 nM as indicated) and LPS (100 ng/ml) during 24 hours.

FIG. 4: shows for Example 4 the kinetics of IL-10 (A), TNF (B) and IL-6 (C) secretion from monocytes co-stimulated with NAT_29 (SEQ ID NO: 1; 1 PM) and LPS (10 ng/ml) during May 10, 2024-48-72 hours (as indicated). Results are presented as fold (NAT_29 vs LPS) and are a mean of six different monocyte donors.

FIG. 5: shows for Example 5 the effects of various mutations in SEQ ID NO: 1/NAT_29 (mutated version: NAT_38/SEQ ID NO: 9). The NAT polypeptides were used at 1 μM, LPS at 100 ng/ml.

FIG. 6: shows for Example 6 the measurement of the transepithelial electrical resistance (TER) (A), histology (H-E staining, B), and IL-8 quantification by ELISA (C) on human ileal resections. Explants were pre-treated 1 hour with NAT_29 (SEQ ID NO: 1) either at 1000 nM or at 10 nM in the apical compartment, then E. coli LF82 was added at 1×10⁹ UFC/ml and incubation was prolonged for 4 hours. Control conditions correspond to explant in media alone. The two upper pictures in (B) show the control without E.coli LF82 (T=0 and T=4 h), while the two lower pictures in (B) show LF82 only and LF82+NAT_29 groups at T=4 h.

FIG. 7: shows for Example 7 in vivo anti-inflammatory properties of NAT29 peptide administered intrarectally in the model of acute colitis induced by TNBS in rats as measured with the Wallace's score. Results are expressed as mean ±SEM score.

FIG. 8: shows for Example 7 in vivo anti-inflammatory properties of NAT29 peptide administered intrarectally in the model of acute colitis induced by TNBS in rats as measured with the lipocalin quantification. Results are expressed as mean ±SEM.

### EXAMPLES

In the following, particular examples illustrating various embodiments and aspects of the invention are presented. However, the present invention shall not to be limited in scope by the specific embodiments described herein. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become readily apparent to those skilled in the art from the foregoing description, accompanying figures and the examples below. All such modifications fall within the scope of the appended claims.

### Example 1: Identification of Human Microbiota-Derived Proteins Inducing IL-10 Release from Human Cells

The aim of this study was to identify proteins expressed by human microbiota, which are able to induce secretion of IL-10 from human immune cells. To this end, a library of proteins expressed by human microbiota was screened to identify proteins inducing secretion of IL-10 from human immune cells.

### Experimental Procedures:

### Library: In Silico Method

A compound library of secreted proteins from gut commensal bacteria was generated by an in silico-based approach. The library included more than 12,000 proteins predicted from human gut microbiome catalogues and from bacterial species known for their role in immune modulation. To obtain the library, bacterial proteins having a length from 50 to 350 amino acids were screened for the presence of secretory signal peptides using the bioinformatic tool Phobius and were annotated using HMMSCAN and the PFAM database. A cut-off at 75% was applied to reduce sequence redundancy.

In view of the relevance of small cysteine-rich proteins in immune modulation an additional selection criterion was applied to identify cysteine-rich proteins: at least two cysteines were required to be present to form a disulphide bond. To ensure correct synthesis and folding in vitro, the amino acid sequences corresponding to the signal peptide were removed.

### Library: Cell Free Proteins Synthesis and Quantification

The protein library was generated using an Escherichia coli Cell Free kit suitable for generation of disulphide bonds (RTS 100 E. coli Disulfide Kit; Biotechrabbit, Hennigsdorf, Germany) according to the supplier's protocol. The Cell-Free system is based on the continuous exchange between the reaction compartment, containing components for transcription and translation, and the feeding chamber, containing amino acids and other energy components, through a semipermeable membrane.

Heterologous protein expression using the transcriptional machinery of E. coli was improved through a codon optimization algorithm (Twist Bioscience, San Francisco, USA) applied to all the selected sequences. All synthetized ORFs were subcloned into pIVEX 2.4 vector (Biotechrabbit, Hennigsdorf, Germany) specifically designed for high-yield Cell-Free expression of His-tagged proteins.

For the detection of His-tagged proteins, the 6His Check kit Gold using the HTRF® technology (Cisbio, Codolet, France) was used according to the supplier's protocol. Proteins, previously diluted at 1:20 in 1X PBS, were quantified in 384 well plates against a standard curve of 6xHis GFP at 0.1μg/mL (ThermoFisher, Waltham, USA) diluted in serial dilution in the lysate used for the Cell-Free synthesis (lysate was also diluted at 1:20 in 1X PBS).

E. coli Production of Recombinant Proteins

DNA from positive hits was subcloned in pET-28a vector carrying an N-terminal 6xHis-Tag (Twist Bioscience, San Francisco, USA) and then transformed in E. coli BL21 (DE3) or Nico21 (DE3) (as in the case of 3166 protein) thermo competent cells (New England Biolabs, Ipswich, MA, USA). For the expression of recombinant proteins, pre-cultures of BL21 (DE3) or Nico21 (DE3) clones were performed in LB-medium at 30° C. under shaking (180 rpm) conditions. Cultures were made in LB or 2YT media under the same conditions and the induction was started when at OD600 of 0.4-0.8 by using 0.1 or 0.5 mM IPTG depending on the protein properties. Induction time was also adapted to each protein and was performed for 2 hours to overnight.

Cultures were centrifuged for 15 min at 4° C., 4500 rpm. Supernatants were removed and the pellets were frozen at −80° C. to break the cells. Pellets were then thawed and resuspended in 1X BugBuster® (Novagen®, Merck KGAA, Darmstadt, Germany) supplemented with Benzonase® Nuclease (Sigma-Aldrich, St. Louis, USA) and Lysozyme (Sigma-Aldrich, St. Louis, USA). Samples were incubated at room temperature by gentle shaking and centrifuged at 4° C., 15,000 g for 30 minutes. Soluble proteins were purified from supernatants onto Nickel packed columns (Protino®, Macherey-Nagel, Düren, Germany) according to the supplier's protocol. In the case of 3166 protein a specific protocol was developed Soluble proteins were purified from supernatants onto HisTrap (Cytivia, Marlborough, USA) using Akta system (Cytivia, Marlborough, USA) following this protocol: Binding Buffer at 50 mM Imidazole, Sample application flow rate at 0,5 mL/min and Elution with mixed mode step: First a gradient of 0 to 25% of Elution Buffer on 5 CV then step at 75% of Elution Buffer on 10 CV then 100% of Elution Buffer on 10 CV. Imidazole, used for proteins elution, was removed by dialysis using 3kDa Slide-A-Lyzer Dialysis Cassettes (ThermoFisher, Waltham, USA). Imidazole, used for proteins elution, was removed by dialysis using 3kDa Slide-A-Lyzer Dialysis Cassettes (ThermoFisher, Waltham, USA). Proteins were visualized on 12% Bis-Tris acrylamide gels (ThermoFisher, Waltham, USA) stained with Coomassie Blue (Imperial protein Stain; ThermoFisher, Waltham, USA) and detected by Western Blot using the 6X-His Tag monoclonal antibody HRP (Miltenyi Biotec, Bergisch Gladbach, Germany) diluted at 1:5000 and revealed using DAB (Sigma-Aldrich, St. Louis, USA).Purified proteins were quantified by Bradford protein assay (Bradford M (1976) A rapid and sensitive method for the quantitation of microgram quantities of protein utilizing the principle of protein-dye binding. Anal. Biochem. 72:248-254).

### IL10 Screening

IL-10 screening of the microbiota protein library was performed on CD14 depleted human peripheral blood mononuclear cells (PBMCs). This cellular model was chosen to reduce the background due to cell wall components and other bacterial contaminants possibly present in the lysate of the Cell Free synthesis kit on the synthesised (but not purified) proteins.

### CD14-Depleted PBMCs:

PBMCs were isolated from buffy coats as follows: 80 ml PBS were added to 50 ml blood; 4 SepMate™-50 IVD tubes (Stemcell Technologies, Vancouver, Canada) were filled with 15 mL of Ficoll® (Ficoll® Paque Plus; Sigma-Aldrich, St. Louis, USA) per donor, then 30 ml of PBS-diluted blood were gently added. Samples were centrifuged for 20 min at 1200 g at room temperature and washed three times with PBS. To lyse the red blood cells, pellets were resuspended in Red Blood Cells Lysis buffer 1X (Miltenyi Biotec, Bergisch Gladbach, Germany) and incubated for 10 min at room temperature. Cells were then washed with MACS buffer and counted.

PBMC depletion was performed using the CD14 Microbeads kit (Miltenyi Biotec, Bergisch Gladbach, Germany) according to the supplier's protocol. Depleted monocytes were resuspended in Iscove's Modified Dulbecco's Medium (IMDM; GIBCO™, Life Technologies, Carlsbad, USA) supplemented with 1% L-Glutamine (Sigma-Aldrich, St. Louis, USA), 1% Penicillin-Streptomycin (Sigma-Aldrich, St. Louis, USA) and 10% of heat-inactivated FBS (Sigma-Aldrich, St. Louis, USA).

### IL-10 Screening:

Screening was performed in 384 well plates in a final volume of 60 μl. PBMCs were seeded at 72,000 cells/well by multidrop and stimulated (Hamilton Robotics, Martinsried, Germany) for 72 hours with 10% (vol/vol) of the library (proteins) previously diluted at 1:10 with PBS in a humidified 5% CO₂ atmosphere at 37° C. The E. coli lysate included in the cell free kit was used (at the same dilution as the library) as negative control. Phytohaemagglutinin (PHA) at 10 g/ml (0.087 μM) was used as positive control. To ensure technical robustness, the screening was performed on CD14 depleted PBMCs from at least two different donors.

IL-10 secretion was measured by AlphaLISA® (IL10 (human) AlphaLISA Detection Kit; PerkinElmer, Waltham MA, USA) on the undiluted supernatants according to the supplier's protocol. Results were expressed as AlphaLISA® signal (Counts). Results were considered as positive hits, either when at least a single raw data signal (of the two signals of the two PBMC donors) was higher than the corresponding plate mean +3SD (Standard Deviation) or when both raw data signals (of the two signals of the two PBMC donors) were higher than the corresponding plate means +2SD (Standard Deviation). To avoid false positives, the concentration of the potential hits obtained by the primary screening was compared to that of the corresponding plate mean.

Potential hits were then validated by a new round of Cell-Free synthesis and test on CD14 depleted PBMCs from several donors (as described above). Further characterization was performed on recombinant proteins produced in the E. coli BL21 (DE3) strain transformed with a pET-28a vector containing the target sequence (as described above, see paragraph “E. coli production of recombinant proteins”). Alternatively, a cell-free production of some proteins was performed by a commercial supplier (Synthelis, La Tronche, France). Peptides were obtained through custom synthesis by a commercial supplier (SB-Peptide or Pepscan).

### Results

### Screening

A total of 11904 proteins of the library was screened on CD14 depleted PBMCs in order to identify proteins able to stimulate IL-10 secretion from human PBMCs. From various potential hits obtained in the primary screening, so far ten proteins were confirmed in the second round of cell-free synthesis. These microbiota proteins, which are able to stimulate IL-10 secretion from human PBMCs, include ID3166 (SEQ ID NO: 2), as well as further microbiota proteins labelled as ID6359; ID1888; ID1889; ID2661; ID5682; ID5138; ID6077; ID6274; and ID6298.

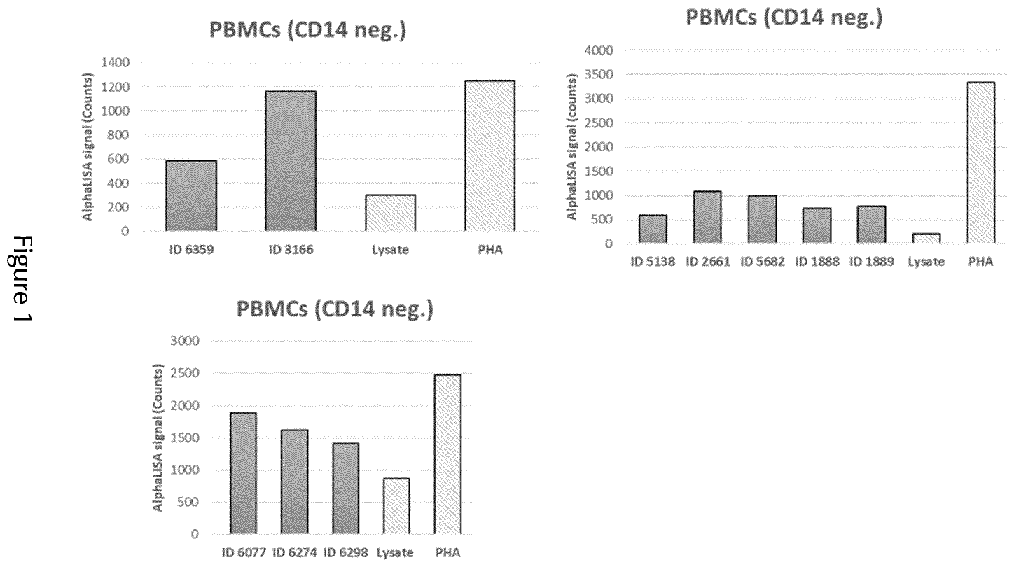

Results of the AlphaLISA for IL-10 secretion from human PBMCs of the second round of cell-free synthesis are shown in FIG. 1. The data show that ten IL-10 secretagogue proteins, including the protein “3166”, were identified by screening of a human microbiome metasecretome protein library on stimulation of IL-10 release from human PBMCs. These microbiota proteins induce IL-10 release from human immune cells.

### Example 2: Dose-Response of Selected IL-10 Inducing Microbiota Proteins

Microbiota proteins ID3166 (SEQ ID NO: 2); ID2661; ID5682; and ID5138 were selected. These proteins contain 121 (ID3166), 144 (ID2661), 58 (ID5682) and 44 (ID5138) amino acids, respectively, corresponding to a size of 14, 15.5, 6 and 4 kDa respectively.

Before testing, the proteins were purified. In view thereof, CD14 depletion of PBMCs was not required, since the background induced by the residual contaminants present in the purified proteins is very low. Therefore, the cellular assays of Example 2 to characterize the purified proteins were performed on total PBMCs.

The selected proteins, were tested at concentrations of 0.5, 0.25, 0.1 and 0.025 μM (final concentration) on PBMCs. Cells were seeded in 384 well plates at 72,000 cells/well in a final volume of 60 pl and stimulated with either the purified proteins (10% vol/vol) or the relative controls (positive and negative) for 24 hours in a humidified 5% CO₂ atmosphere at 37° C. Samples were tested in duplicate on at least two different PBMCs donors. All the dilutions were made in PBS.

Secretion of IL-10 was measured by AlphaLISA and compared to those of PBS (negative control) and PHA at 10 μg/ml (positive control), essentially as described above.

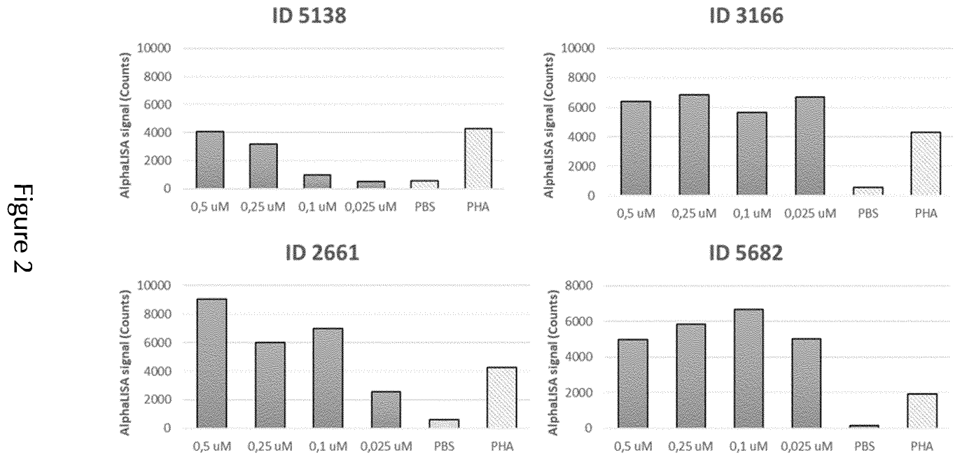

Results of the AlphaLISA for IL-10 secretion from human PBMCs at different doses of the selected microbiota proteins are shown in FIG. 2. The data show that all the tested proteins are as effective (ID5138) as PHA (positive control) or even more effective (ID 3166; ID2661; and ID5682) than PHA in inducing IL-10 secretion from PBMCs. Moreover, microbiota protein ID3166 shows very high effectivity even at the lowest dose tested.

### Example 3: In-vitro Characterization of an IL-10 Inducing Fragment of Microbiota Protein ID3166

A 28-amino-acid fragment of microbiota protein ID3166 (referred to as “NAT_29”; SEQ ID NO: 1) was investigated with regard to its IL-10 inducing/enhancing capacity. The NAT_29 sequence (SEQ ID NO: 1) is characterized by two cysteines (C7 and C23) and, without being bound to any theory, the present inventors assume that it forms a loop that may share similarities with a class of human hormones.

To investigate the IL-10 inducing/enhancing capacity of NAT_29, in vitro IL-10 secretion of human immune cells in the presence of different doses of NAT_29 was tested. Briefly, human CD14+ monocytes were isolated from peripheral blood mononuclear cells (PBMCs) from healthy human volunteers by density gradient centrifugation using SepMate™-50 IVD tubes (Stemcell, Ref. 85460). The obtained PBMCs were washed thrice with Dulbeccos's phosphate buffered saline (PBS, Sigma, D88537) and lysed 10 min at room temperature in Red blood Lysis buffer 1X (Miltenyi Biotec, 130-094-183). Cells were washed with MACS buffer (Miltenyi Biotec, 130-091-221) and counted using the trypan blue dye exclusion method. CD14+monocytes were sorted using anti-human CD14 Microbeads (Miltenyi Biotec, 130-050-201) as per manufacturer's protocol. Positive CD14+ fractions were collected and seeded at 2×10⁴ cells per well in 384 well plates in IMDM medium (Gibco, 12440-046) supplemented with 10% FBS and 1X antibiotic solution.

Monocyte-derived dendritic cells (MoDCs) were derived from the differentiation of CD14+ cells, obtained after the selective sorting. CD14+ cells are cultured for 7 days at the density of 1.5×10⁶ cells/mL in complete IMDM medium supplemented with 20ng/ml of Human IL4 (Miltenyi Biotec, 130-093-921) and 20ng/ml of Human GM-CSF (Miltenyi Biotec, 130-093-865). MoDCs were seeded at 1.2 x 104 cells per well in 384 well plates in IMDM medium.

Monocytes or MoDCs were stimulated with various concentrations (from 1 to 1000 nM) of NAT_29 and co-stimulated with 100 ng/ml of LPS from E. coli O111: B4 (InvivoGen, tlrl-eblps) and placed in humidified CO₂ incubator at 5% CO₂/37° C. for 24 h. IL-10 secretion were measured at 24 hours by AlphaLISA® on cell supernatants according to the supplier's protocol and read on an Envision plate reader (Perkin Elmer).

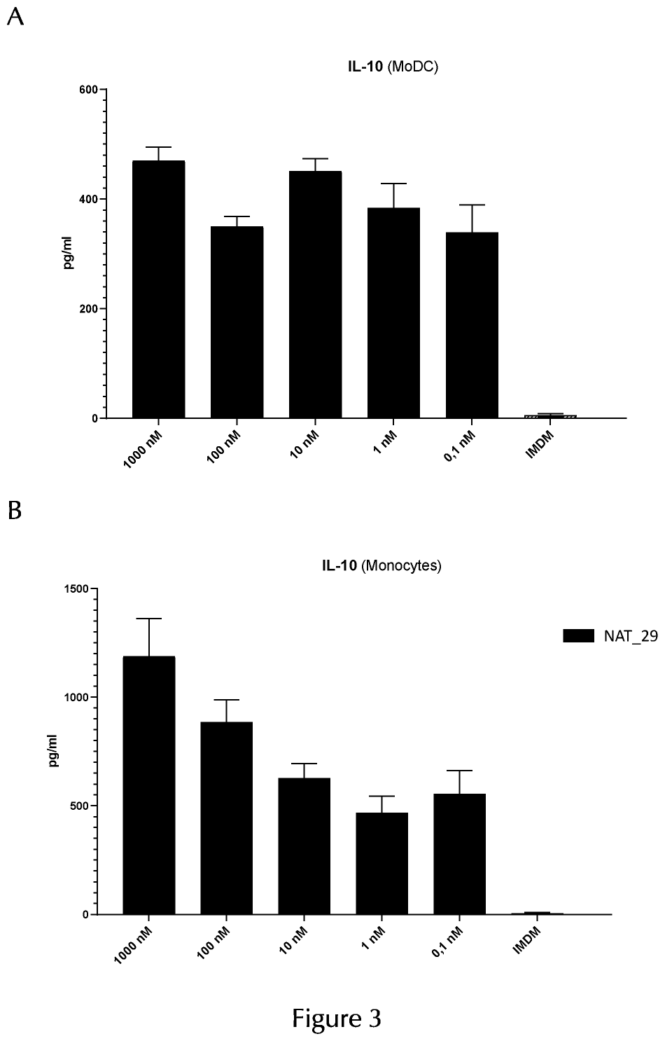

Results are shown in FIG. 3 (A: MoDCs; B: monocytes). The results demonstrate an IL-10 secretagogue activity of NAT_29 (SEQ ID NO: 1). NAT_29 was tested within a dose range between 0.1 and 1000 nM and highest IL-10 levels were measured in both cell types at the highest concentration of NAT_29 tested, but even at considerable lower concentrations of NAT_29, IL-10 secretagogue activity can still be observed. In this assay, monocytes appeared to be more sensitive than MoDCs to the action of NAT_29. For this reason, further in-vitro characterization of NAT_29 was performed on monocytes.

### Example 4: NAT 29 has Anti-Inflammatory Properties in Vitro

To confirm the anti-inflammatory potential of NAT_29 (SEQ ID NO: 1), a kinetic study was performed to test the concomitant secretion of IL-10 and the major pro-inflammatory cytokines TNF-α and IL-6. To this end, monocytes were obtained as described in Example 3 and stimulated with 1 μM NAT_29 polypeptide (SEQ ID NO: 1), co-stimulated with 10 ng/ml of LPS from E. coli O111: B4 (InvivoGen, tlrl-eblps) and placed in humidified CO₂ incubator at 5% CO₂/37° C. for 72 h. IL-10, TNF-α and IL-6 secretion were measured at 5, 10, 24, 48 and 72 hours by AlphaLISA® on cell supernatants according to the supplier's protocol (Perkin, ref. AL218F, AL208F, AL223F) and read on an Envision plate reader (Perkin Elmer).

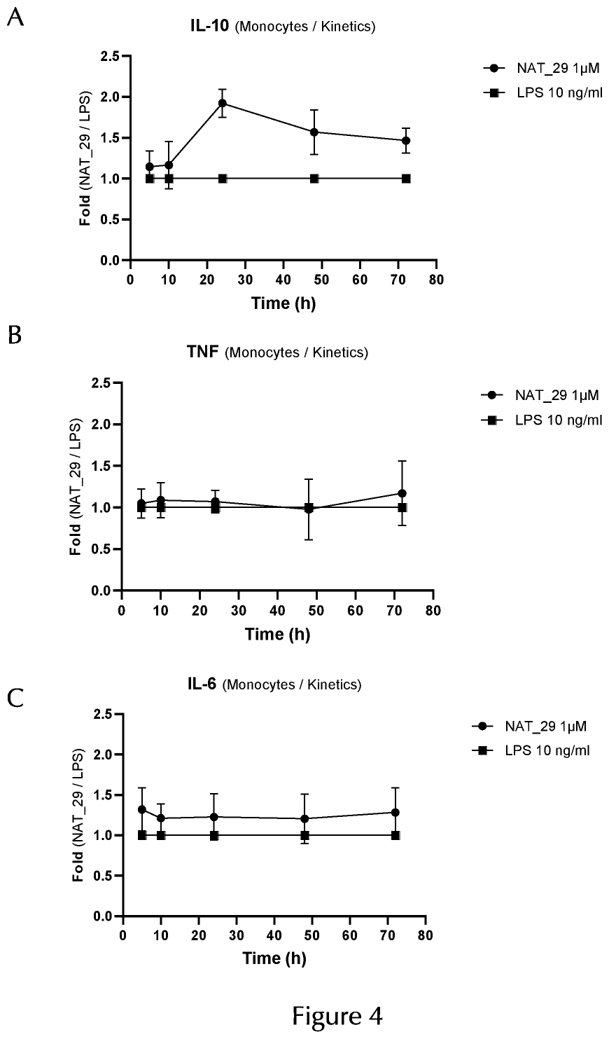

Results are shown in FIG. 4 (A: IL-10; B: TNF-a; C: IL-6). This kinetic study confirmed the IL-10 secretagogue activity of NAT_29 (SEQ ID NO: 1) and showed a peak of IL-10 secretion at 24 h reaching a fold of two (2) in respect to LPS (FIG. 4A). IL-10 secretion remained constant (at 1,5-Fold) until the end of the experiment (72 h).

In contrast thereto, no significant secretion of the major pro-inflammatory cytokines TNF-a and IL-6 was measured throughout the experiment (FIGS. 4B-C). Overall, these data demonstrate a positive balance of IL-10 secretion vs secretion of pro-inflammatory cytokines induced by the NAT_29 polypeptide.

### Example 5: Various Mutations in NAT 29 Do Not Abolish IL-10 Secretion

In order to investigate the impact of different sequence variations of NAT_29, monocytes from six donors were obtained as described in Example 3 and stimulated with 1 μM NAT_29 polypeptide (SEQ ID NO: 1) or a sequence variant thereof containing eight mutations (NAT_38; SEQ ID NO: 9), co-stimulated with 100 ng/ml LPS (NAT_38 comparison) from E. coli O111: B4 (InvivoGen, tlrl-eblps) and placed in humidified CO₂ incubator at 5% CO2/37° C. for 24 h. IL-10 secretion was measured at 24 hours by AlphaLISA® on cell supernatants according to the supplier's protocol and read on an Envision plate reader (Perkin Elmer).

For comparison with NAT_29 (SEQ ID NO: 1), a polypeptide differing from the NAT_29 sequence by eight (8) amino acid positions was chosen (NAT_38; SEQ ID NO: 9). Namely, in SEQ ID NO: 9
(i) 110 of SEQ ID NO: 1 is deleted,
(ii) D13 in SEQ ID NO: 1 is substituted with P (D13P),
(iii) a Q is added between R14 and G15 of SEQ ID NO: 1,
(iv) T16 in SEQ ID NO: 1 is substituted with Y (T16Y),
(v) K17 in SEQ ID NO: 1 is substituted with Q (K17Q),
(vi) S22 in SEQ ID NO: 1 is substituted with G (S22G),
(vii) an I is added between F25 and Y26 of SEQ ID NO: 1, and
(viii) P28 of SEQ ID NO: 1 is deleted.

These differences are illustrated in the below comparison of the sequences:

(SEQ ID NO: 1)
KKGSRSCFFIYLDR-GTKKGSRSCFF-YLP
(SEQ ID NO: 9)
KKGSRSCFF-YLPRQGYQKGSRGCFFIYL-

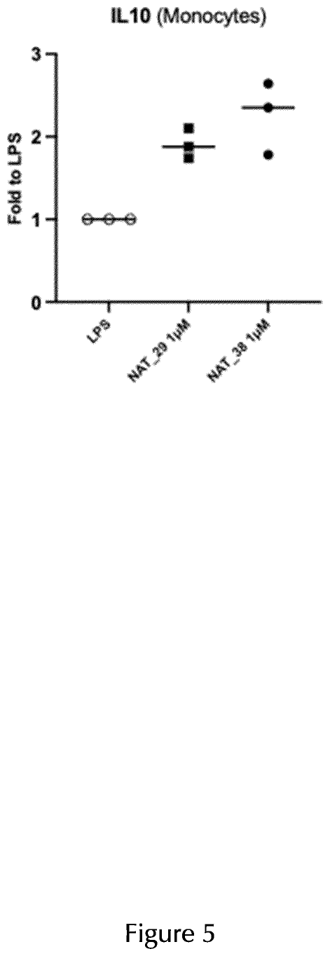

Results are shown in FIG. 5 (A: various mutations (NAT_38); B: C-terminal truncations (NAT_31.1, NAT_31.2 and NAT_31.3). These data show that despite the various mutations introduced into the sequence of NAT_29 (SEQ ID NO: 1) in the polypeptide NAT_38 (SEQ ID NO: 9), the IL-10 secretagogue activity was maintained. Accordingly, the IL-10 secretagogue activity does not strictly depend on the exact sequence of NAT_29 (SEQ ID NO: 1), but tolerates various mutations.

### Example 6: NAT 29 has Anti-Inflammatory Properties Ex Vivo and INCREASES the integrity of Epithelial Cells after Inflammation

Next, the role of NAT_29 was evaluated in the context of inflammatory diseases using ex vivo experiments performed on human ileal resections. To this end, explants were mounted on Ussing Chambers in order to separate the apical side from the basolateral side as naturally occurs in the gut. Tissues were pre-incubated 1 h with NAT_29 (SEQ ID NO: 1) before inflammation was triggered with live Escherichia coli LF82 during 4 hours.

Preparation of Bacteria: The day before the assay, LB broth (Sigma Aldrich, L3522) was inoculated, using sterile loops, with bacteria from a glycerol stock (E. coli LF82-gfp) maintained at −80° C. Tubes were incubated overnight at 37° C. without shaking. The next day, optical density of the bacterial suspensions was read at 600 nm using a spectrophotometer (Amersham Ultrospec 10) allowing estimation of the bacterial cell density and bacteria were added to human explants as explained below.

### Preparation of Human Ileal Explants:

Human tissue samples were obtained from patients undergoing surgery with the patients' agreement. Samples were taken from macroscopically unaffected area as identified by the surgeon. After resection, the specimens were placed in ice-cold oxygenated DMEM media (Gibco, 61965-026) containing 1% streptomycin/penicillin solution (Gibco, 15140-122) and 50 μg/mL gentamicin (Thermofisher, Gentamicin Hyclone, SV30080-03). Intestinal resections were extensively washed and were cleaned from vascular vessels and conjunctive tissue using forceps under binocular microscope. Muscularis and plexus were removed and only the epithelial layer was kept. Intestinal explants were then isolated from the cleaned resections using surgical punch (diameter of 1 cm2). Intestinal explants were washed three times in large volumes of DMEM media without antibiotics before being mounted in Ussing chambers (Harward apparatus, 66-0015) allowing access to either apical/luminal or basolateral/serosal sides of the explants. Both compartments of the Ussing chambers were filled with 1 ml of DMEM media without antibiotics before exposure to NAT_29 and bacteria.

### Exposure of Human Ileal Explants to Fractions and Bacteria:

Human ileal explants were left untreated or were pre-treated (apical/mucosal compartment of the Ussing's chambers) for 1 h at 37° C. with NAT_29 at 10 nM or 1 μM. After the 1 h pre-incubation period at 37° C. in a CO₂ incubator (Panasonic, model MCO-19AICUV-PE) LF82-gfp bacteria were added into the apical compartment of the Ussing chambers at 1×10⁹ bacteria/mL together with NAT_29 at 10 nM or 1 μM.

Three parameters were assessed to evaluate the anti-inflammatory potential of NAT_29 in the ex vivo model:
1) Transepithelial Electrical Resistance (TER) which is a measure of integrity/tightness of the human explants;
2) histology;
3) IL-8 secretion (quantified by Elisa).

### Transepithelial Electrical Resistance (TER) Assay

TER assays are well-known methods for measuring effects on the structural and functional integrity of an epithelial cell layer, which are described, for example, in: Srinivasan et al., 2015, J Lab Autom, 20:107-126. Accordingly, the integrity/tightness of the human explants during the incubation was evaluated through Transepithelial Electrical Resistance (TER) measurement using Millicell-ERS (Electrical Resistance System) Voltohmmeter (Millipore, MERS00002). Supernatant aliquots from the basolateral side were collected at TO and T4 h for IL-8 measurement.

### IL-8 Analysis:

IL-8 levels were measured by ELISA (BD Biosciences, 555244). ELISA were revealed using the HRP substrate Sigma Fast OPD (Sigma-Aldrich, P9187-50SET). Absorbances were measured using a microplate reader (BioTek, Synergy MX, model SMATLD) at 490 nm.

### Microscopy (H&E Staining)

At the end of the experiment, human explants were removed from Ussing chambers, washed 6-times with 2 mL of PBS (Gibco, 14040-091) and fixed using PBS PFA 4% solution (Merck, ZC906196547) at 4° C. during 24 h. The day after, all punches were washed twice with 1ml of PBS. Punches were then cut in two and included in inclusion medium (TFM-EMS, 72592), in transverse position to allow cutting in the crypt-villosity axis. Four sections of 5 μm thickness were obtained per explant, each section being separated by 100 um from the next in order to cover all the tissue (cryostat Leica CM3050). Explants were then stained using Hematoxylin and Eosin (H&E) Staining Protocol.

The procedure was as follows:
incubation 8 min in Hematoxylin (Sigma-Aldrich, HS16-500mL)
incubation 2 min in tap water (to allow stain to develop)
incubation 1 min in eosin (Sigma-Aldrich, 318906-500)
incubation 1 min in tap water
incubation 2 min in 70% ethanol
incubation 2 min in 95% ethanol
incubation 2 min in 100% ethanol
incubation 15 min in xylene (performed twice)
mounting with coverslip slides using Eukitt (Histological mounting medium)
drying overnight.

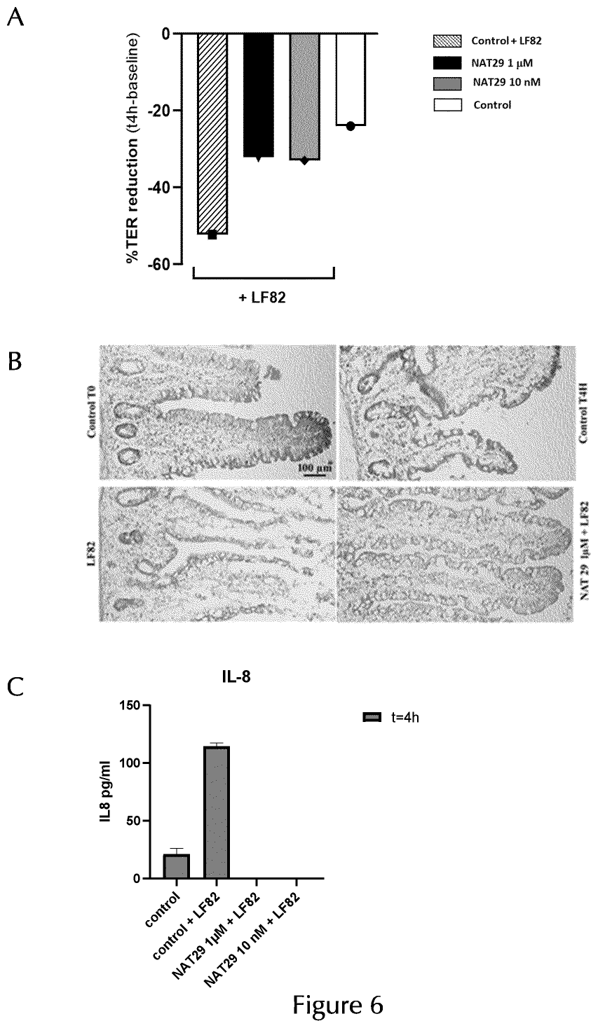

Results are shown in FIG. 6 (A: TER assay; B: histology; C: IL-8 secretion). As observed in FIG. 6A, explants incubated during 4 hours with E. coli LF82 alone showed a marked decrease in TER (52% reduction). Pre-incubation of tissues with NAT_29 (SEQ ID NO: 1) either at 1000 nM or 10 nM similarly prevented the degradation of the tissue as observed by TER value (32% reduction) that was similar to the control condition without inflammation triggered by E. coli LF82 (24% reduction).

TER results were then confirmed by histological analysis, as shown in FIG. 6B. The observations of the H&E staining (FIG. 6B) showed that at T=0, the intestinal mucosa was perfectly homogeneous without desquamation. The thickness of the mucosa was normal, reflecting a non-inflammatory baseline condition of the tissue. For the T=4 h control without bacterial infection, a thinner mucosa especially in the crypts was observed, indicating a pre-inflammatory stage of the tissue probably due to the stress experienced by the tissue during chamber assembly and incubation at 37° C. for 4 hours. Incubation for 4 hours in the presence of LF82 bacteria induced an advanced inflammatory state with a mucosa totally desquamated at its apical pole and also at the level of the villus. Explant pre-incubation with 1000 nM NAT_29 induced a protective effect against LF82-induced degradation. Under this condition both, mucosa and sub-mucosa, remain similar to the control at the same time point (T =4 h).

Quantification of the pro-inflammatory cytokine IL-8 from media recovered from the basolateral side of the Ussing Chamber confirm the overall anti-inflammatory potential of NAT_29. As shown in FIG. 6C, IL-8 showed 4-fold increase following incubation with LF82. Treatment with NAT-29 at either 1000 or 10 nM completely abolished IL-8 secretion.

In summary, these data demonstrate the anti-inflammatory potential of NAT_29 as well as its advantageous effects on the structural and functional integrity of the epithelial cell layer in an ex vivo model of an inflammatory disease.

### Example 7: NAT 29 Polypeptide has Anti-Inflammatory Properties in Vivo

Next, anti-inflammatory properties of the NAT_29 polypeptide (SEQ ID NO: 1) have been evaluated in the 2,4,6-trinitrobenzene sulfonic acid (TNBS)-induced colitis model. The model of colitis induced by TNBS in rats is a validated animal model to evaluate the anti-inflammatory properties of drugs in intestinal bowel diseases (IBD). TNBS induces deep inflammation resulting in necrosis (with transmural destruction of the intestine wall) of the colon. This model is well-characterized, reliable, reproducible, and admitted by regulatory authorities in IBD (e.g., as described in Antoniou, E., et al. (2016). The TNBS-induced colitis animal model: An overview. Annals of medicine and surgery, vol. 11, 9-15, which is incorporated herein in its entirety).

To this end, the NAT_29 polypeptide (SEQ ID NO: 1) was administered by intrarectal instillation at two different concentrations (1 and 0.1 μM) in the model of acute colitis induced by TNBS (intrarectal administration at 80 mg/kg) in Sprague-Dawley rats. The anti-inflammatory effects were evaluated at the macroscopic level using validated score of Wallace and compared to those induced by the positive control (5-ASA) administered also by intrarectal instillation.

Resolution of inflammation was also evaluated using colon Lipocalin-2 (Lcn-2) quantification. Lipocalin-2, also known as neutrophil gelatinase-associated lipocalin (NGAL), is considered as a clinically relevant biomarker of IBD. Lipocalin 2 is expressed in normal tissues and is markedly increased when inflammation occurs, such as in colonic epithelial tissue from IBD patients. In rodent models of colitis, a strong association has been shown between elevated levels of Lcn-2 and colitis severity.

### A. Materials and Methods

Male Sprague-Dawley rats (Body weight: around 100-150 g) were allocated randomly and blindly 4 per cage and acclimated for one week. Rats were obtained from Janvier Laboratoires, Le Genest Saint-Isle, France.

The tested products were administered once a day by intrarectal instillation (500μl) in a prophylactic and therapeutic mode starting at day -5, i.e. 5 days before colitis induction (DO) and until euthanasia at D4, corresponding to 9 treatment days in total.

For colitis induction, Sprague Dawley rats were anesthetized for 2 hours using a subcutaneous injection of xylazine at 12.5 mg/kg/ketamin at 25 mg/kg. Colitis was induced by an intrarectal injection of 250 μL TNBS (80 mg/kg in 40% EtOH) at 8 cm from the anus using a catheter. The TNBS solution at 80 mg/kg was prepared based on the mean body weight of rats, which is recorded before the TNBS solution preparation.

Five animal groups were used for the study, which are shown in Table 2 below.

TABLE 2
experimental groups

| | | | | Number |
|---|---|---|---|---|
| | | | | of rats/ |
| Test Articles | Dose | | Administration | groups |
| Control + Vehicle (No TNBS) | Not applicable | | Intrarectal | 5 |
| TNBS + Vehicle | Not applicable | | Intrarectal | 10 |
| TNBS + NAT29 | 0.1 | μM | Intrarectal | 10 |
| TNBS + NAT29 | 1 | μM | Intrarectal | 10 |
| TNBS + Positive control | 30 | mM | Intrarectal | 10 |
| Pentasa ® | | | | |

NAT_29 was solubilized in PBS and the active concentration for animal treatment was calculated assuming a maximum volume of colon-rectum content in rat of 3.5ml. To achieve a final concentration of 0.1 μM in the rat colon, 500 μl of a 0.7 μM solution of NAT_29 was administrated intrarectally. To achieve a final concentration of 1μM in the rat colon, 500 μl of a 7 μM solution of NAT_29 was administrated intrarectally.

Pentasa®, was used as a positive control of an anti-inflammatory compound at a final concentration of 30 mM. Evaluation of the intensity of colitis was performed 4 days later.

Animals were sacrificed 4 days (D4) after TNBS administration (DO) for evaluation of the intensity of colitis. Samples from distal colon were collected and used for macroscopic assessments and lipocalin quantification.

Macroscopic and histological assessments of colitis were performed independently by two operators to validate the score. The colon of each rat was examined to evaluate the macroscopic lesions according to the Wallace criteria. The Wallace score rates macroscopic lesions on a scale from 0 to 10, and this score is based on features reflecting inflammation, such as hyperemia, thickening of the bowel, and extent of ulceration, as shown in Table 3 below.

TABLE 3
Wallace score

| Score | Criteria of macroscopic evaluation |
|---|---|
| 0 | No Inflammation |
| 1 | Hyperemia without ulcerations |
| 2 | Hyperemia with thickenning of the mucosa without ulcerations |
| 3 | 1 ulceration without thickenning of the colonic wall |
| 4 | 2 or more of ulcerative or inflammatory sites |
| 5 | 2 or more ulcerative and inflammatory sites with an extent >1 cm |
| 6 | Ulcerative or inflammatory site >2 cm |
| 7 | Ulcerative or inflammatory site >3 cm |
| 8 | Ulcerative or inflammatory site >4 cm |
| 9 | Ulcerative or inflammatory site >5 cm |
| 10 | Ulcerative or inflammatory site >6 cm |

The level of Lipocalin-2 (Lcn-2), a marker of neutrophil infiltration (enzyme contained in polymorphonuclear neutrophil primary granules) was quantified by ELISA (Clinisciences, Rat Neutrophil Gelatinase Associated Lipocalin NGAL ref DLR-NGAL-Ra-96T) on a piece of distal colon taken off at euthanasia. Colon pieces were homogenized (50 mg/ml) in Tris-HCI buffer containing protease inhibitors (Sigma-Aldrich) in a Precellys homogenizer using ceramic beads (1.4 & 2.8 mm) (Bertin, France).

All comparisons were analyzed using the Permutation Test for two independent samples using StatXact software. The comparisons were done against the TNBS control group. Differences were considered statistically significant if the p value was <0.05.

### B. Results

Evaluation of anti-inflammatory effects of the tested products was performed 4 days after the colitis induction by TNBS. This step corresponds to the end of the peak of inflammation. No mortality related to the administration of the tested products, or the induction of colitis, was recorded, thereby indicating the safety and the innocuity of daily rectal administration of the active products, even under inflammatory conditions, during 8 consecutive days.

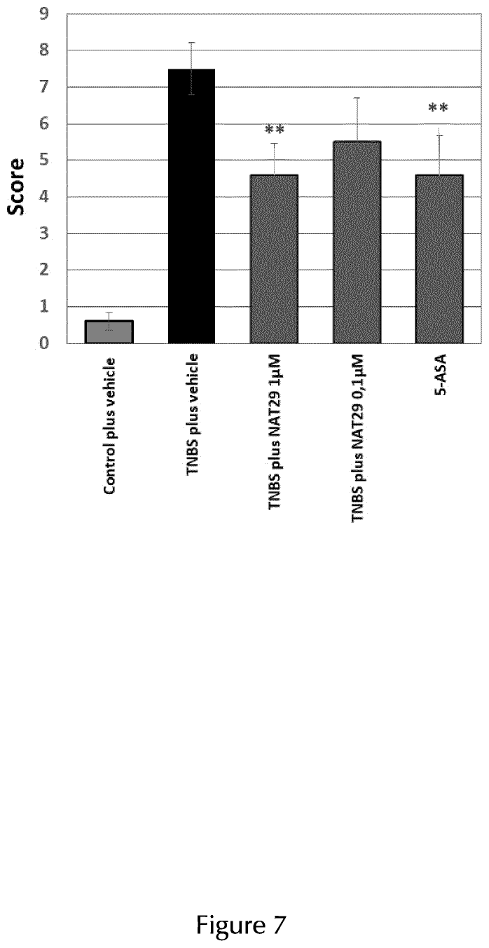

Intensity of colonic inflammation and lesions was evaluated at macroscopic level according to the validated Wallace's criteria as described in Table 3. Results are shown in FIG. 7. The results show that intrarectal administration of NAT_29 at a final concentration of 1 μM elicit a statistically protective effect on inflammatory parameters as measured with the Wallace score. The level of protection is similar to the one observed with the reference compound 5-ASA (Pentasa). Treatment with NAT_29 at a final concentration of 0.1 μM also elicited a protective effect (albeit not significant).

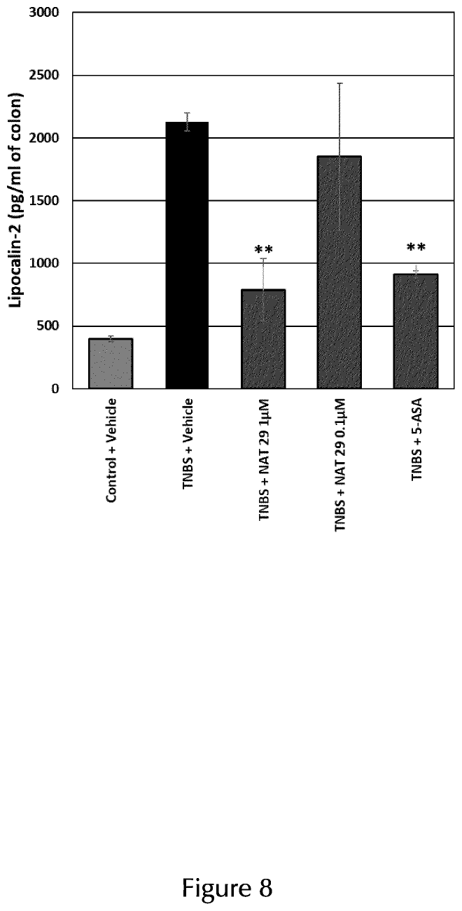

Intensity of colonic inflammation was evaluated using lipocalin quantification (ELISA) per ml of colonic tissue. Lipocalin levels are considered as a biomarker of disease severity mainly secreted by neutrophils, being modulated in serum, faecal sample, and colonic samples from animal models of colitis as in human suffering from IBDs. Results are shown in FIG. 8. The results show that intrarectal administration of NAT_29 at a final concentration of 1UM is associated with a significant decrease of Lipocalin levels in the colon. The level of protection is similar to the one observed with the reference compound 5-ASA (Pentasa). Treatment with NAT_29 at a final concentration of 0.1 μM is associated with a slight decrease of lipocalin-2 level (albeit not significant).

TABLE OF
SEQUENCES AND SEQ ID NUMBERS (SEQUENCE LISTING):

| SEQ ID NO | Sequence | Remarks |
|---|---|---|
| SEQ ID NO: 1 | KKGSRSCFFIYLDRGTKKGSRSCFFYLP | NAT_29 |
| SEQ ID NO: 2 | AFLFTSTGVPKKAAEAAFFLYLNKGTKK | ID3166 |
| | GSRSCFFIYLDRGTKKGSRSCFFYLPRQ | |
| | GYQKGSRGCFFIYLDRGTKKGSRGCFFI | |
| | YLDCEKRAGNVCIRKCRGRYLHKKTPRR | |
| | YRNAEATCS | |
| SEQ ID NO: 3 | SCXXYLP | NAT_29 motif |
| SEQ ID NO: 4 | SCXXXYLD | NAT_29 motif |
| SEQ ID NO: 5 | SCFFYLP | NAT_29 motif |
| SEQ ID NO: 6 | KKGSRSCFFYLP | NAT_29 motif |
| SEQ ID NO: 7 | SCFFIYLD | NAT 29 motif |
| SEQ ID NO: 8 | KGSRSCFFIYLD | NAT_29 motif |
| SEQ ID NO: 9 | KKGSRSCFFYLPRQGYQKGSRGCFFIYL | NAT_38 |
| SEQ ID NO: 10 | AAAAAAGGCAGCAGAAGCTGCTTTTTTA | NAT_29 |
| | TTTACCTCGACAGGGGTACCAAAAAAGG | |
| | CAGCAGAAGCTGCTTTTTTTATTTACCT | |

1. A polypeptide comprising or consisting of an amino acid sequence according to SEQ ID NO: 1, wherein, optionally, 0, 1, 2, 3, 4, 5, 6, 7 or 8 amino acids may be substituted, added and/or deleted, with the proviso that the serine residue at position 6 of SEQ ID NO: 1 is maintained.

2. The polypeptide according to claim 1, wherein the polypeptide does not comprise an amino acid sequence according to SEQ ID NO: 2.

3. The polypeptide according to claim 1 or 2, wherein the cysteine residue(s) at position 7 and/or 23 of SEQ ID NO: 1 is/are maintained.

4. The polypeptide according to any one of the previous, wherein the polypeptide has a length of no more than 120 amino acids.

5. A polypeptide comprising or consisting of an amino acid sequence according to general formula (I):
(I)
SCX₁X₂X₃YLX₄
wherein
X₁ may be any amino acid,
X₂ may be any amino acid,
X₃ may be any amino acid or is deleted, and
X₄ is P or D.

6. The polypeptide according to claim 5 comprising or consisting of an amino acid sequence according to general formula (Ia):
(Ia)
KGSRSCX₁X₂X₃YLX₄
wherein X₁-X₄ are defined as in claim 5.

7. The polypeptide according to claim 5 or 6, wherein X₃ is deleted and X₄ is P, preferably wherein the polypeptide comprises an amino acid sequence according to SEQ ID NO: 5 or 6.

8. The polypeptide according to claim 5 or 6, wherein X₃ may be any amino acid and X₄ is D, preferably wherein the polypeptide comprises an amino acid sequence according to SEQ ID NO: 7 or 8.

9. The polypeptide according to any one of the previous claims comprising two cysteine residues, optionally forming a disulfide bond.

10. A polypeptide comprising or consisting of an amino acid sequence according to general formula (II):
(II)
CX₁X₂X₃YLX₄X₅X₆X₇X₈X₉KGSRX₁₀C
wherein
X₁ may be any amino acid,
X₂ may be any amino acid,
X₃ may be any amino acid or is deleted,
X₄ is Por D,
X₅ may be any amino acid,
X₆ may be any amino acid or is deleted,
X₇ may be any amino acid,
X₈ may be any amino acid,
X₉ is K or Q, and
X₁₀ may be any amino acid.

11. The polypeptide according to any one of the previous claims comprising or consisting of an amino acid sequence according to general formula (Ila):
(IIa)
SCFFX₃YLX₄RX₆GX₈X₉KGSRX₁₀C
wherein
X₃ is I or deleted,
X₄ is Por D,
X₆ is Q or deleted,
X₈ is T or Y,
X₉ is K or Q, and
X₁₀ is S or G.

12. The polypeptide according to claim 10 or 11, wherein either X₃ or X₆, but not X₃ and X₆, are deleted.

13. The polypeptide according to any one of claims 5 to 12 having a length of at least 17 amino acids.

14. The polypeptide according to any one of claims 1 to 4 comprising an amino acid sequence as defined in any one of claims 5 to 13.

15. The polypeptide according to any one of the previous claims, wherein the polypeptide does not comprise an amino acid sequence according to SEQ ID NO: 2.

16. The polypeptide according to any one of the previous claims, wherein the polypeptide has a length of no more than 120 amino acids.

17. The polypeptide according to any one of the previous claims, wherein the polypeptide is capable of inducing and/or enhancing IL-10 secretion from human cells.

18. The polypeptide according to any one of the previous claims, wherein the polypeptide comprises or consists of an amino acid sequence according to SEQ ID NO: 1, wherein the polypeptide does not comprise an amino acid sequence according to SEQ ID NO: 2.

19. The polypeptide according to any one of the previous claims, wherein the polypeptide comprises or consists of an amino acid sequence according to SEQ ID NO: 9.

20. A nucleic acid comprising a polynucleotide encoding the polypeptide according to any one of the previous claims.

21. The nucleic acid according to claim 20, wherein the nucleic acid is a DNA molecule or an RNA molecule; preferably selected from genomic DNA; cDNA; siRNA; rRNA; mRNA; antisense DNA; antisense RNA; ribozyme; complementary RNA and/or DNA sequences; RNA and/or DNA sequences with or without expression elements, regulatory elements, and/or promoters; a vector; and combinations thereof.

22. The nucleic acid according to claim 20 or 21, wherein the nucleic acid sequence of the polynucleotide encoding the polypeptide, shares at least 80% sequence identity with SEQ ID NO: 10.

23. The nucleic acid according to any one of claims 20 to 22, wherein the polynucleotide encoding the polypeptide is codon-optimized for expression by prokaryotic cells, preferably bacteria.

24. An expression cassette comprising a polynucleotide encoding the polypeptide according to any one of claims 1 to 19 and, operably linked thereto, a regulatory element, preferably for expression in a prokaryotic cell, such as a bacterium.

25. The expression cassette according to claim 24 comprising a regulatory element for heterologous expression and/or overexpression of the encoded polypeptide.

26. A vector comprising the nucleic acid according to any one of claims 20 to 23 or the expression cassette according to claim 24 or 25.

27. A (host) cell expressing the polypeptide according to any one of claims 1 to 19; or comprising the nucleic acid according to any one of claims 20 to 23, the expression cassette according to claim 24 or 25, or the vector according to claim 26.

28. The (host) cell according to claim 27, wherein the (host) cell is a bacterium, preferably a genetically engineered bacterium.

29. A genetically engineered bacterium capable of inducing and/or enhancing IL-10 secretion from a human cell, the bacterium comprising the nucleic acid according to any one of claims 20 to 23, the expression cassette according to claim 24 or 25, or the vector according to claim 26.

30. A genetically engineered bacterium (over) expressing the polypeptide according to any one of claims 1 to 19.

31. A culture medium comprising the polypeptide according to any one of claims 1 to 19, the (host) cell according to claim 27 or 28, or the bacterium according to claim 29 or 30.

32. The culture medium according to claim 31 further comprising a (self) antigen.

33. An isolated human cell cultured with the culture medium according to claim 31 or 32.

34. The cell according to claim 33, wherein the cell is a human immune cell, preferably a PBMC.

35. The cell according to claim 33 or 34, wherein the cell is a monocyte, a macrophage, a dendritic cell or a lymphocyte, preferably a T lymphocyte.

36. A pharmaceutical composition comprising the polypeptide according to any one of claims 1 to 19, or the nucleic acid according to any one of claims 20 to 23, the vector according to claim 26, the cell according to claim 27 or 28, the bacterium according to claim 29 or 30, or the human cell according to any one of claims 33 to 35, and, optionally, a pharmaceutically acceptable excipient or carrier.

37. The polypeptide according to any one of claims 1 to 19, the nucleic acid according to any one of claims 20 to 23, the vector according to claim 26, the cell according to claim 27 or 28, the bacterium according to claim 29 or 30, the human cell according to any one of claims 33 to 35, or the pharmaceutical composition according to claim 36 for use in medicine.

38. The polypeptide, the nucleic acid, the vector, the cell, the bacterium, the human cell or the pharmaceutical composition for use according to claim 37 in treating an inflammatory disease or an autoimmune disorder.

39. The polypeptide, the nucleic acid, the vector, the cell, the bacterium, the human cell or the pharmaceutical composition for use according to claim 37 in treating inflammatory bowel disease (IBD).

40. The polypeptide, the nucleic acid, the vector, the cell, the bacterium, the human cell or the pharmaceutical composition for use according to claim 37 in treating an allergy.

41. A method for reducing, treating, alleviating symptoms of or ameliorating an inflammatory disease or an autoimmune disorder in a subject, comprising the step of administering to said subject the polypeptide according to any one of claims 1 to 19, the nucleic acid according to any one of claims 20 to 23, the vector according to claim 26, the cell according to claim 27 or 28, the bacterium according to claim 29 or 30, the human cell according to any one of claims 33 to 35, or the pharmaceutical composition according to claim 36.

42. A method for inducing and/or enhancing IL-10 secretion in a subject, comprising the step of administering to said subject the polypeptide according to any one of claims 1 to 19, the nucleic acid according to any one of claims 20 to 23, the vector according to claim 26, the cell according to claim 27 or 28, the bacterium according to claim 29 or 30, the human cell according to any one of claims 33 to 35, or the pharmaceutical composition according to claim 36.

43. A method for inducing tolerance in a subject, comprising the step of administering to said subject the polypeptide according to any one of claims 1 to 19, the nucleic acid according to any one of claims 20 to 23, the vector according to claim 26, the cell according to claim 27 or 28, the bacterium according to claim 29 or 30, the human cell according to any one of claims 33 to 35, or the pharmaceutical composition according to claim 36.
